(19) United States
(12) Patent Application Publication
(10) Pub. No.: US 2026/0290567 A1
(43) Pub. Date: Sep. 24, 2026

(54) METHODS AND SYSTEMS FOR PATIENT DISCHARGE MANAGEMENT

(71) Applicant: GE Precision Healthcare LLC, Waukesha, WI (US)

(72) Inventors: Jehoshua Chang, Atlanta, GA (US); Jeffrey Terry, Dallas, TX (US); Bex Thomas, San Ramon, CA (US); Kathleen Martin, Pinckney, MI (US)

(21) Appl. No.: 19/693,239

(22) Filed: May 29, 2026

Related U.S. Application Data
(63) Continuation-in-part of application No. 17/804,940, filed on Jun. 1, 2022, now Pat. No. 12,658,309.

(51) Int. Cl. G16H 40/20; G16H 10/60; G16H 40/40; G16H 50/70; G16H 80/00

(52) U.S. Cl. CPC G16H 40/20; G16H 10/60; G16H 40/40; G16H 50/70; G16H 80/00

(57) ABSTRACT

A patient discharge date prediction system generates probabilistic discharge timing predictions using a trained length of stay prediction model that receives patient data and operational workflow data encoded into data vectors. The model simultaneously outputs a predicted discharge date, a discharge horizon probability distribution indicating probabilities of discharge on a specified date, the next day, or two or more days from the specified date, and a time-of-day discharge probability distribution indicating probabilities of discharge within specific time windows during the day. Wilson score interval calibration is applied to the probability distributions based on historical observed discharge rates to generate calibrated probabilities and confidence intervals. The calibrated probabilities and confidence intervals are displayed in a graphical user interface. The system automatically initiates discharge preparation workflows when predicted probabilities exceed configurable thresholds, and updates predictions as new patient data becomes available throughout the hospital stay.

### FIELD

Embodiments of the subject matter disclosed herein relate to managing patients of a healthcare facility, and more particularly, to monitoring patient status.

### BACKGROUND

A demand for resources of a healthcare facility (e.g., beds, ventilators, etc.) may be high, to provide appropriate care to a continuous stream of patients with serious medical conditions. An important responsibility of care providers working in a healthcare facility is determining when patients may be discharged, to free up resources of the healthcare facility for new patients. An average length of stay for patients with a same condition as the patient (e.g., a geometric mean length of stay, or GMLOS) may be provided, for example, within a patient manager software application. However, the GMLOS may not be accurate, as a result of not taking into consideration specific health issues of the patient. An expected discharge date (EDD) of a patient may also be provided in the patient manager software application by a physician. However, the EDD may not be accurate due to an amount of time taken by the physician to determine an accurate EDD. Reviewing patient records in an EHR system to determine whether a patient may be ready to discharge may be laborious and time consuming. The patient data to be reviewed may include numerous medical procedures and records generated during investigations of the patient, including a variety of examinations such as blood tests, urine tests, pathology reports, image-based scans, etc. The data may be stored in an Electronic Health Record (EHR) system, which may aggregate information from a plurality of different computer systems and databases. Sorting and extracting information may be a slow and inefficient process, increasing a likelihood of missing records with relevant data which may be spread out across a large number of less informative records.

As a result, an accurate determination of when a patient may be discharged may not be made with sufficient advance notice to efficiently manage patient discharges. The patient discharges may be delayed due to preparations such as filing paperwork, arranging transportation, and other administrative tasks not being performed in a timely manner. Delayed patient discharges may reduce an efficiency in resource allocation within the healthcare facility.

Existing patient discharge prediction systems suffer from a further limitation in that they provide only a single predicted discharge date or a date window, without indicating the relative likelihood that a patient will be discharged on any particular day within or near that window. A clinician or case manager reviewing a predicted discharge date of “tomorrow” has no way of knowing, from the prediction alone, whether that prediction reflects a high-confidence near-certainty or a marginally probable outcome among several equally plausible alternatives. Similarly, existing systems do not indicate whether a patient predicted for discharge on a given day is more likely to be discharged in a first time interval, a second time interval, or a third time interval during the day. For example, the first time interval may correspond to a morning shift, the second time interval may correspond to an afternoon shift, and the third time interval may correspond to an evening shift. This absence of intra-day discharge timing information creates operational inefficiencies in hospital workflows that depend on discharge timing, including bed management, transport scheduling, pharmacy preparation, and downstream placement coordination. Without probabilistic discharge horizon and time-of-day information, care teams cannot prioritize their discharge-related activities across a patient census in a manner that reflects the actual likelihood of discharge occurring within any given time window.

A further limitation of existing systems is that they do not provide calibrated statistical confidence measures around their discharge predictions. Even where a system outputs a predicted discharge date or window, it does not communicate the degree of statistical confidence associated with that prediction, nor does it validate that confidence against historical observed discharge rates. As a result, clinicians and administrators have no reliable basis for distinguishing predictions that are well-supported by the underlying data from predictions that carry substantial uncertainty. What is needed is a system and method that generates probabilistic discharge horizon predictions across multiple discharge timing categories, generates time-of-day discharge probability distributions, and applies a statistically rigorous calibration methodology to express confidence bounds around those predictions in a manner that has been validated against historical discharge outcomes.

### BRIEF DESCRIPTION

In one embodiment, a method for a patient management system of a healthcare facility comprises receiving a selected confidence level from a user of the patient management system; predicting a date for discharging a patient of the healthcare facility using a trained length of stay (LOS) prediction model, based on a set of patient data of the patient, the LOS prediction model trained on historical patient data of the healthcare facility; predicting a discharge date window of the patient, based on the received confidence level; generating a predicted discharge date window element summarizing the predicted discharge date window in a user interface (UI) of the patient management system; and displaying the UI on a display device of the patient management system.

In another embodiment, a method for a patient management system of a healthcare facility comprises receiving patient data of a patient of the healthcare facility from one or more data sources; generating, using a trained patient length-of-stay prediction model, a discharge horizon probability distribution over possible discharge times; generating, using the trained patient length-of-stay prediction model, a time-of-day discharge probability distribution indicating a probability that the patient will be discharged within a first time window on a day, a probability that the patient will be discharged within an second time window on the day, and a probability that the patient will be discharged within a third time window on the day; applying Wilson score interval calibration to the discharge horizon probability distribution and the time-of-day discharge probability distribution based on historical observed discharge rates to generate calibrated probabilities and associated confidence intervals for each probability; and displaying the calibrated discharge horizon probabilities, the calibrated time-of-day discharge probabilities, and the associated confidence intervals in a user interface of the patient management system.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a first data flow diagram showing an exemplary patient discharge date prediction system, in accordance with an aspect of the disclosure;

FIG. 2 is a schematic block diagram showing components of an exemplary patient discharge date prediction system, in accordance with an aspect of the disclosure;

FIG. 3 shows an example user interface of a patient management system including the patient discharge date prediction system of FIG. 2, in accordance with an aspect of the disclosure;

FIG. 4 shows a timeline along which patient data may be generated, in accordance with an aspect of the disclosure;

FIG. 5A shows a patient data schematic where different types of patient data are stacked based on a timeline, in accordance with an aspect of the disclosure;

FIG. 5B shows a data vector comprising encoded patient data, in accordance with an aspect of the disclosure;

FIG. 6A shows a block diagram of a first exemplary embodiment of a patient length of stay (LOS) prediction model training system, in accordance with an aspect of the disclosure;

FIG. 6B shows a block diagram of a patient LOS prediction model in use during deployment, in accordance with an aspect of the disclosure;

FIG. 7 shows a flowchart illustrating an exemplary method for training a CNN-based LOS prediction model to predict a length of stay for a patient, in accordance with an aspect of the disclosure;

FIG. 8 shows a flowchart illustrating an exemplary method for deploying a trained CNN-based LOS prediction model to generate a predicted length of stay for a patient, in accordance with an aspect of the disclosure;

FIG. 9A is a second data flow diagram of the patient discharge date prediction system, illustrating the flow of patient data inputs through a patient LOS prediction model to generate a predicted length of stay, a discharge horizon probability distribution, and a time-of-day probability distribution;

FIG. 9B is a block diagram illustrating the flow of predicted discharge date, calibrated horizon probabilities and confidence intervals, and calibrated time-of-day probabilities and confidence intervals into a patient manager GUI display;

FIG. 10 is a screenshot of an exemplary display panel of a patient manager graphical user interface illustrating discharge horizon probability information and time-of-day discharge probability information;

FIG. 11 is a diagram of an exemplary confidence interval display element showing a point estimate, a lower confidence bound, an upper confidence bound, and a graphical confidence range indicator;

FIG. 12 is a flowchart of an exemplary method for generating and displaying calibrated patient discharge probability predictions;

FIG. 13 is a flowchart of an exemplary method for generating Wilson score interval confidence bounds for patient discharge probability predictions;

FIG. 14A shows a block diagram of a second exemplary embodiment of a patient length of stay (LOS) prediction model training system; and

FIG. 14B shows a block diagram of the second exemplary embodiment of the patient LOS prediction model in use during deployment.

### DETAILED DESCRIPTION

The following description relates to systems and methods for managing patients in a hospital or healthcare organization. Efficient patient management may include predicting when patients will be discharged. By accurately predicting patient discharge dates, care providers may efficiently allocate resources (e.g., beds, ventilators, etc.) in preparation for new patients with serious conditions.

Patient data may be stored in one or more electronic health record (EHR) systems. Different types of patient data may be stored in different locations, accessed via different interfaces, and used in different ways to make decisions about patient care. The medical data may include diagnostic data, pathology data, consultation data, and treatment data, which may be accessed in different ways. In some examples, a care provider may consult diagnostic data by accessing a first system, pathology data by accessing a second system, consultation data by accessing a third system, and treatment data by accessing a fourth system. In other examples, the diagnostic data, pathology data, consult data, and treatment data may be accessible via a single EHR system, but a user may have to perform a different set of actions (e.g., entering in search terms, selecting controls, etc.) from different reference points to view each different type of data. Viewing and considering such data independently when making patient care decisions may lead to overlooked information that may affect a treatment decision, and may increase time burden and mental load placed on the clinicians caring for a patient.

As disclosed herein, the issues described above may be addressed by an AI-based, computerized patient discharge date prediction system that automatically retrieves and aggregates data relevant to health conditions of a plurality of patients, and generates predicted discharge dates for the plurality of patients. The predicted discharge dates may be used to generate predicted date windows for discharging patients, at varying levels of confidence. For example, a first discharge date window may be a first range of dates during which a patient has an estimated 90% chance of being discharged within the first discharge date window. A second discharge date window may be a second range of dates during which a patient has an estimated 60% chance of being discharged within the second discharge date window. As a result of having a lower confidence level (e.g., 60% vs 90%), the second range of dates of the second discharge date window may be a narrower range of dates than the first range of dates of the first discharge date window.

The automatic generation and display of patient predicted discharge date windows (also referred to herein as discharge window predictions) may reduce an amount of time spent by a care provider in determining statuses of a rotating body of patients, thereby increasing an amount of time available to the care provider to attend to patients. Further, a cognitive load placed on the care provider may also be reduced, thereby reducing a probability of error in assessing when a patient may be discharged.

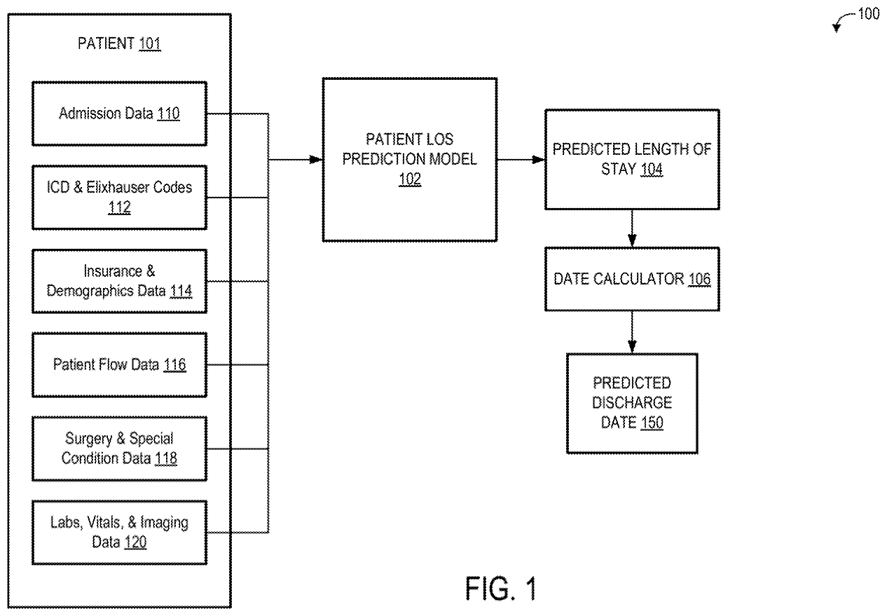

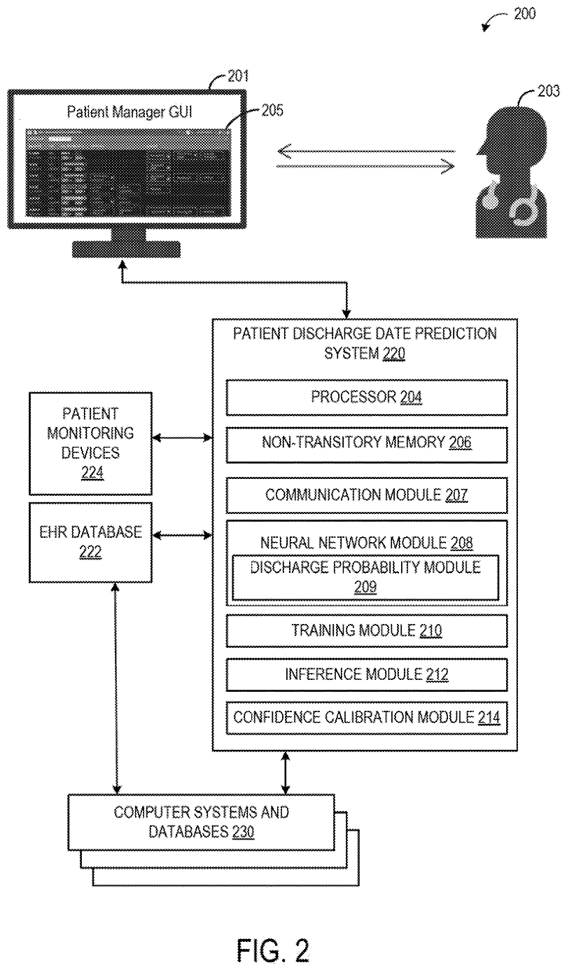

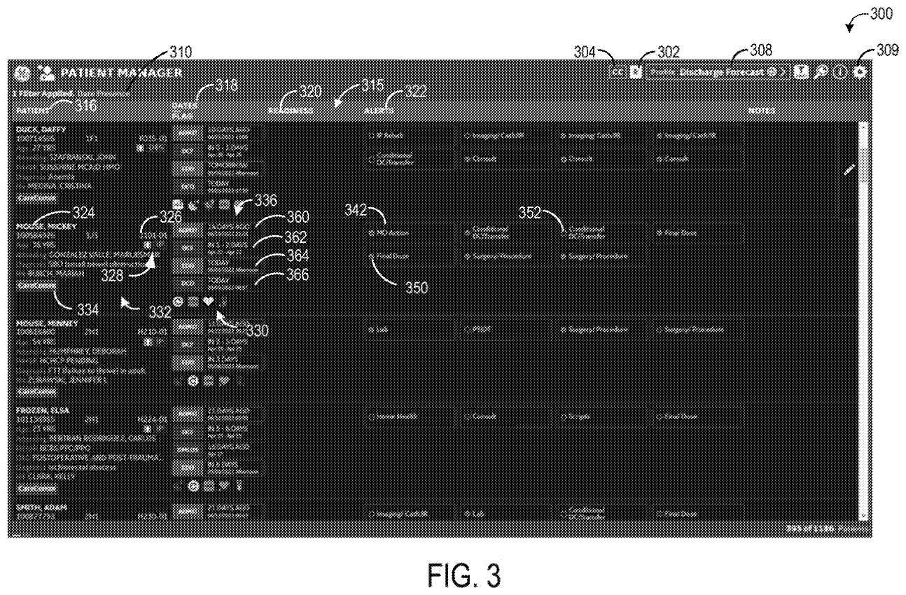

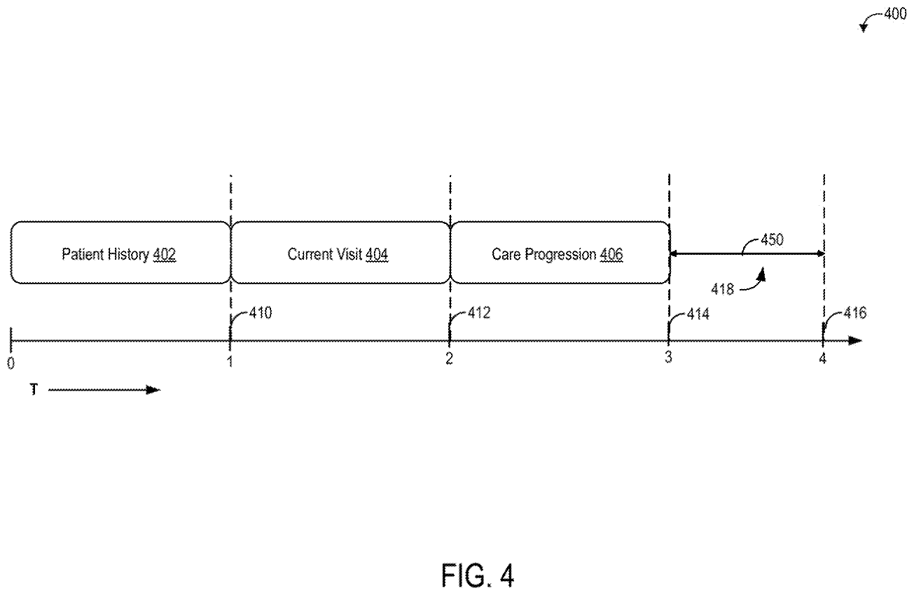

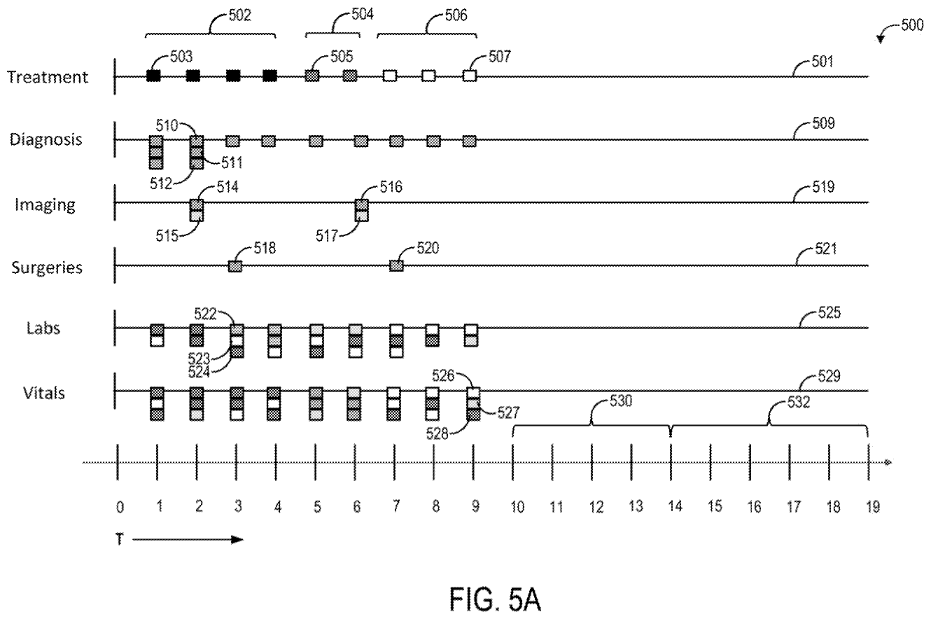

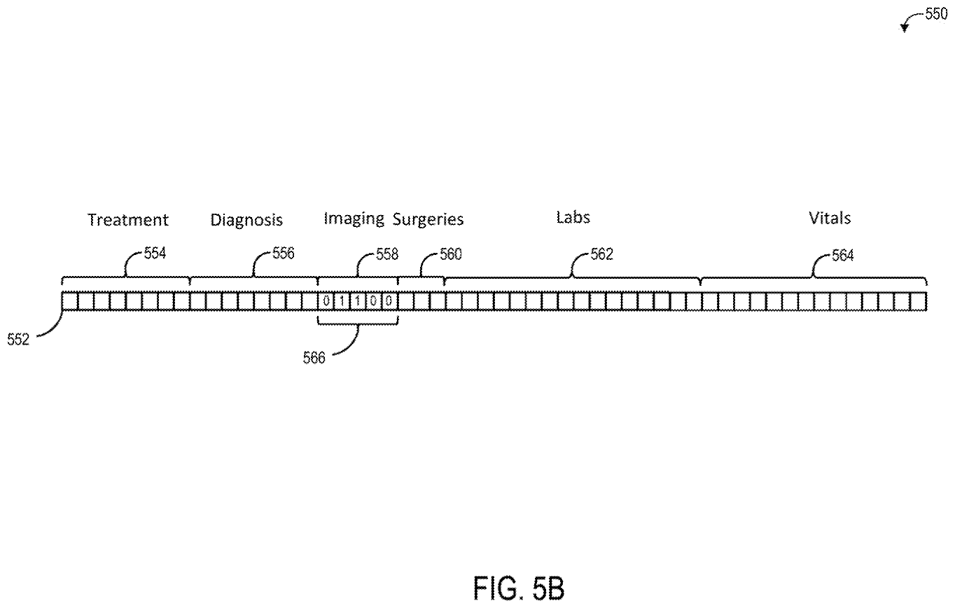

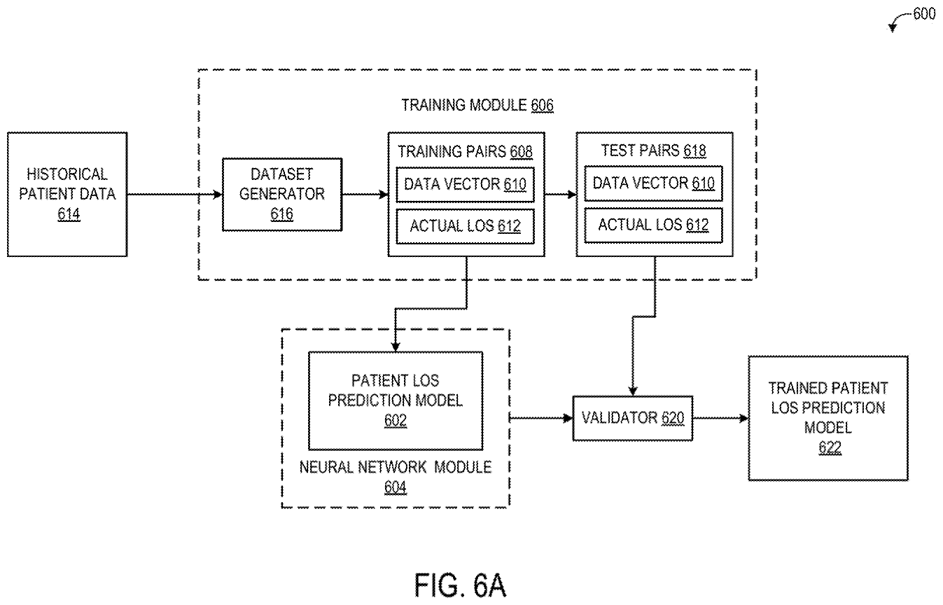

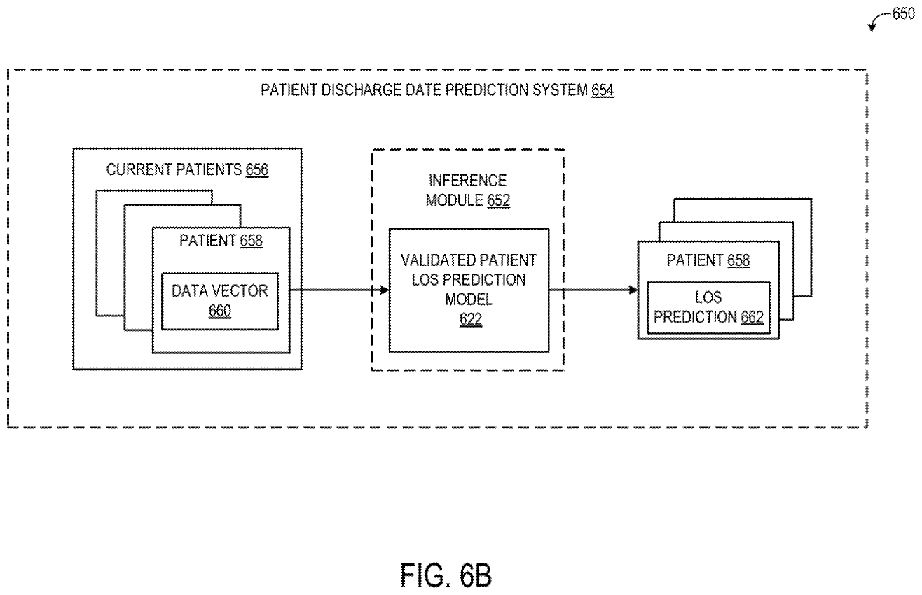

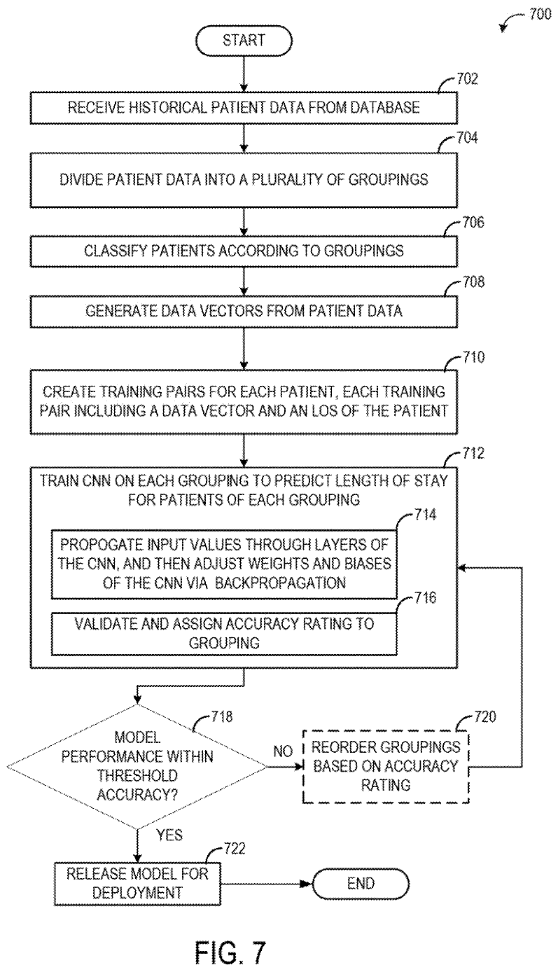

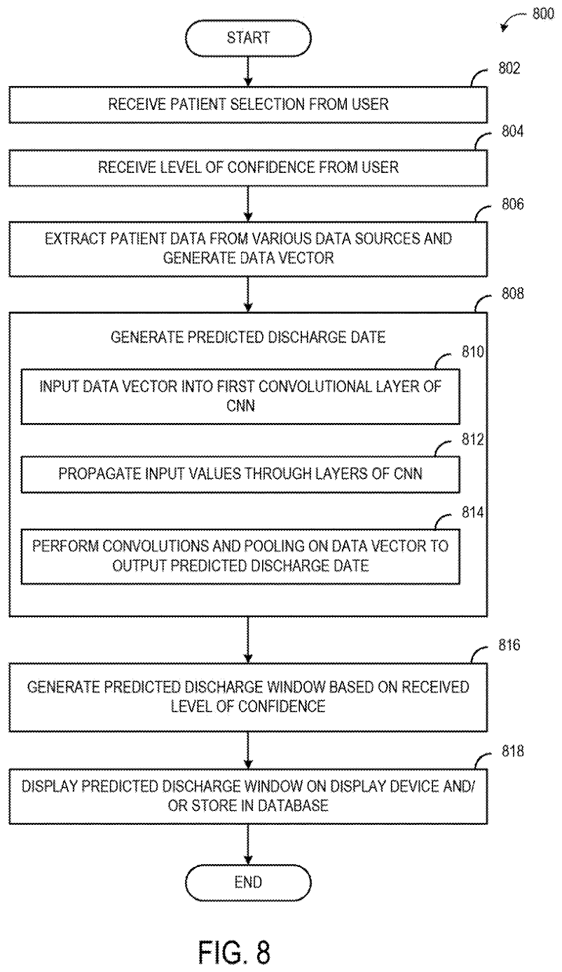

Embodiments of the present disclosure will now be described, by way of example, with reference to the figures. FIG. 1 shows a flow of data through an exemplary patient discharge date prediction system, such as the patient discharge date prediction system of FIG. 2. The patient discharge date prediction system may generate a graphical element indicating a predicted discharge date, or discharge date window, in a user interface (UI) of a patient manager web application, such as the UI of FIG. 3. The patient data used to predict the discharge date may be generated over a patient timeline, as shown in FIG. 4. Prior to being processed, the patient data may be organized and/or structured based on a stacked approach of FIG. 5A, from which a data vector comprising encoded patient data may be created, as shown in FIG. 5B. The data vectors of encoded patient data may be used to train a patient length of stay (LOS) prediction model, based on the LOS prediction model training system shown in FIG. 6A. The LOS prediction model may be trained by following one or more steps of the method shown in FIG. 8. FIG. 6B shows the patient LOS prediction model in use during deployment, where the LOS prediction model may generate a predicted length of stay of a patient by following one or more steps of the method shown in FIG. 7. The patient discharge date prediction system may then convert the length of stay prediction to a predicted date of discharge, or discharge date window, which may be displayed in the UI.

Referring now to FIG. 1, a flow of data through an exemplary patient discharge date prediction system 100 of a hospital network is shown. Patient discharge date prediction system 100 includes a patient LOS prediction model 102, which may predict an amount of time a patient 101 is expected to stay (e.g., length of stay) in one or more hospitals of the hospital network. The expected stay of patient 101 may be predicted from a plurality of patient data of patient 101, which may be received from a plurality of EHR's and/or databases of the hospital network.

For example, patient discharge date prediction system 100 may receive one or more secure feeds of patient data, where as a result of the patient data being included within the hospital network, a security of the data may be established whereby the patient data may be received without using encryption and/or authentication. In various embodiments, the secure feeds may be generated by patient discharge date prediction system 100, where patient discharge date prediction system 100 may automatically and/or regularly request patient data from the plurality of EHR's and/or databases of the hospital network. For example, patient discharge date prediction system 100 may request a first set of data from a first EHR of the hospital network, and receive a first secure feed from the first EHR including the first set of data; patient discharge date prediction system 100 may request a second set of data from a second EHR of the hospital network, and receive a second secure feed from the second EHR including the second set of data; and so on.

The one or more secure feeds may include patient data of various types. For example, the patient data received in the one or more secure feeds may include admission data 110 of patient 101. Admission data 110 may include information pertaining to an admission of patient 101 into a hospital of the hospital network, such as a date and time of admission, a health condition and/or symptoms for which patient 101 was admitted, a primary care provider of patient 101, contact information of patient 101, and the like. The patient data may include International Classification of Diseases (ICD) and Elixhauser Comorbidity Index codes 112, which may indicate a disease or health condition of patient 101. The patient data may include insurance and demographics data 114. The patient data may include patient flow data 116, which may indicate a trajectory of patient 101 through a healthcare system such as the healthcare system offered by the hospital network. For example, the trajectory may include prior diagnoses, treatments, and stays at one or more hospitals of the hospital network. The patient data may include surgery and special condition data 118, which may indicate one or more surgeries performed on patient 101, as well as outcomes of the surgeries and any discoveries made during the surgeries. The patient data may include labs, vital sign data, and imaging data 120, which may include results from tests, studies, and/or panels performed on patient 101, data received at one or more EHRs of the hospital network from patient monitors arranged at or on patient 101, and/or results of computed tomography (CAT), magnetic resonance (MR), and/or other imaging studies performed on patient 101. It should be appreciated that the examples provided herein are for illustrative purposes, and other types of patient data may be included in one or more secure feeds without departing from the scope of this disclosure.

Patient LOS prediction model 102 may receive the patient data from patient 101 as input, and may output a predicted LOS 104. For example, the predicted LOS 104 may be a number of days that patient 101 is expected to remain a patient of a hospital of the hospital network, based on historical patient data used to train the patient LOS prediction model 102. Predicted LOS 104 may be an input into a date calculator 106 of the patient discharge date prediction system 100, which may calculate a predicted discharge date 150 of patient 101 based on predicted LOS 104 and a current date.

Thus, according to embodiments disclosed herein, a patient discharge date prediction system may be employed to retrieve and compile relevant patient status information from a variety of sources, and automatically generate and display discharge date predictions for patients at a hospital. The discharge date predictions may facilitate an efficient and timely preparation for discharging patients from the hospital. The generation and display of the discharge date predictions may reduce a footprint of the patient medical data and may reduce the cognitive load on the user/clinician.

FIG. 2 shows a patient management system 200 according to an aspect of the disclosure. Patient management system 200 may include a patient discharge date prediction system 220 configured for predicting a discharge date window of a patient that may be displayed, along with associated clinical information, to a user 203 via a display 201. Display 201 may be any known device having a screen to display the discharge window prediction and the clinical information. The clinical information may be stored in and/or may be retrieved from one or more computer systems or databases 230 of patient management system 200. The clinical information may be generated during investigations of the patient (e.g., clinical visits, examinations, tests) and may include a variety of medical records including examination reports, blood tests, urine tests, pathology reports, and scans using various medical imaging systems such as ultrasound, magnetic resonance imaging, computed tomography systems, and other radiological investigations. These medical records generated from various investigations may be stored in different formats. For example, the medical images may be stored in a digital imaging in medical communication (DICOM) format, which is different from a format for storing the blood parameter reports, which is different from the format of storing the pathological reports, and so on.

Display device 201, and in some examples, more than one display device, may be communicatively coupled to patient discharge date prediction system 220. Display device 201 may include a processor, memory, communication module, user input device, display (e.g., screen or monitor), and/or other subsystems and may be in the form of a desktop computing device, a laptop computing device, a tablet, a smart phone, or other device. Display device 201 may be adapted to send and receive encrypted data and display medical information, including medical images in a suitable format such as digital imaging and communications in medicine (DICOM) or other standards. Display device 201 may be located locally at a medical facility, or the display device may be a mobile device accessed remotely from the medical facility (such as a care provider's mobile phone).

The patient discharge window predictions may be viewed via a user interface (UI) 205 (which may be a graphical user interface or GUI) of display device 201. When viewing the patient discharge window predictions via UI 205, a care provider may enter input (e.g., via a user input device, which may include a keyboard, mouse, microphone, touch screen, stylus, or other device) that may be processed by patient discharge date prediction system 220. In examples where the user input is a selection of a link or control button of UI 205, the user input may trigger display of additional clinical information relevant to one or more patients, or other actions.

Patient management system 200 may include an electronic health record (EHR) database 222, which may be communicatively coupled to patient discharge date prediction system 220. EHR database 222 may be stored in a mass storage device configured to communicate with secure channels (e.g., HTTPS and TLS), and store data in an encrypted format. Further, EHR database 222 may be configured to control access to patient electronic medical records such that only authorized healthcare providers may edit and access the electronic health records. An EHR for a patient may include medical device data (e.g., vital signs measured or otherwise ascertained by medical devices such as heart rate, oxygen saturation, etc.), user-specified medical parameters (e.g., acuity scores, pain scores), patient demographic information, family medical history, past medical history, lifestyle information, preexisting medical conditions, current medications, allergies, surgical history, past medical screenings and procedures, past hospitalizations and visits, and/or other information.

In addition to EHR database 222, the patient information used to generate the discharge window predictions may also be stored in one or more computer systems and databases 230 in communication with patient discharge date prediction system 220 and/or EHR database 222. For example, computer systems and databases 230 may include a picture archiving and communication system (PACS) that may store and communicate medical images and associated reports (e.g., clinician findings), such as ultrasound images, MRI images, etc., in the DICOM format. Computer systems and databases 230 may include a radiology information system (RIS), which may store radiology images and associated reports, such as CT images, X-ray images, etc. Computer systems and databases 230 may include a pathology database, which may store pathology images and related reports, including visible light or fluorescence images of tissue, such as immunohistochemistry (IHC) images. In some embodiments, the patient information used to generate the discharge window predictions may also be received from one or more patient monitoring devices 224 communicably coupled to patient discharge date prediction system 220. It should be appreciated that the example computer systems and databases provided herein are for illustrative purposes, and in various embodiments, additional, fewer, or different computer systems and/or databases may be included without departing from the scope of this disclosure.

Patient discharge date prediction system 220 may include one or more processors 204 and a non-transitory memory 206 to store and execute the methods disclosed herein to generate the discharge window predictions and patient status data, as well as send and receive communications, graphical user interfaces, medical data, and other information. Non-transitory memory 206 may include one or more data storage structures, such as optical memory devices, magnetic memory devices, or solid-state memory devices, for storing programs and routines executed by processor(s) 204 to carry out various functionalities disclosed herein. Non-transitory memory 206 may include any desired type of volatile and/or non-volatile memory such as, for example, static random access memory (SRAM), dynamic random access memory (DRAM), flash memory, read-only memory (ROM), etc. Processor(s) 204 may be any suitable processor, processing unit, or microprocessor, for example. Processor(s) 204 may be a multi-processor system, and, thus, may include one or more additional processors that are identical or similar to each other and that are communicatively coupled via an interconnection bus. Processor(s) 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processors 204 may be virtualized and executed by remotely-accessible networked computing devices configured in a cloud computing configuration.

In some examples, patient discharge date prediction system 220 may be implemented over a cloud or other computer network. For example, patient discharge date prediction system 220 is shown in FIG. 2 as constituting a single entity, but it should be appreciated that patient discharge date prediction system 220 may be distributed across multiple devices, such as across multiple servers.

Patient discharge date prediction system 220 may be configured to generate and output one or more patient discharge window predictions for display via display 201. Each patient discharge window prediction may include additional clinical information, which may be automatically extracted from the EHRs and/or other medical information of the patient (e.g., pathology reports, imaging scans/reports, etc.) that may or may not be stored as EHRs. In some embodiments, a portion of the extracted clinical information may be assembled into a natural-language-like format and displayed along with and/or adjacent to a relevant discharge window prediction, as described in greater detail below in reference to FIG. 3. For example, the extracted clinical information may include doctor's notes, radiologist notes to assess a condition of a patient, and/or other factors to use as features in machine/deep learning.

As used herein, the term “module” may include a hardware and/or software system that operates to perform one or more functions. For example, a module may include a computer processor, controller, or other logic-based device that performs operations based on instructions stored on a tangible and non-transitory computer readable storage medium, such as a computer memory. The device may be an off-the-shelf device that is appropriately programmed or instructed to perform operations described herein from the instructions described above. Alternatively, a module may include a hard-wired device that performs operations based on hard-wired logic of the device. Various modules shown in the attached figures may represent the hardware that operates based on software or hardwired instructions, the software that directs hardware to perform the operations, or a combination thereof.

Patient discharge date prediction system 220 may include a communication module 207. Communication module 207 may facilitate transmission of electronic data within and/or among one or more systems. Communication via communication module 207 may be implemented using one or more protocols. In some examples, communication via communication module 207 occurs according to one or more standards (e.g., Digital Imaging and Communications in Medicine (DICOM), Health Level Seven (HL7), ANSI X12N, etc.). Communication module 207 can be a wired interface (e.g., a data bus, a Universal Serial Bus (USB) connection, etc.) and/or a wireless interface (e.g., radio frequency, infrared, near field communication (NFC), etc.). For example, communication module 207 may communicate via wired local area network (LAN), wireless LAN, wide area network (WAN), etc. using any past, present, or future communication protocol (e.g., BLUETOOTH™, USB 2.0, USB 3.0, etc.).

Patient discharge date prediction system 220 may include a neural network module 208. Neural network module 208 may store one or more patient discharge window prediction models that may be used to generate the discharge window predictions. The patient discharge window prediction models may include one or more machine learning (ML) models and/or deep learning models (e.g., neural networks), and instructions for implementing the one or more patient discharge window prediction models to predict a patient discharge window, as described in greater detail below. Neural network module 208 may include trained and/or untrained neural networks and may further include various data, or metadata pertaining to the one or more neural networks stored therein.

Neural network module 208 may include a discharge probability module 209 within neural network module 208. Discharge probability module 209 may include instructions and model parameters for generating probabilistic discharge timing predictions for patients of the healthcare facility. In particular, discharge probability module 209 may generate, for each patient, a discharge horizon probability distribution over possible discharge times. For example, the discharge horizon probability distribution may indicate a probability that the patient will be discharged on a specific date, such as a current date, a probability that the patient will be discharged the day after the specific date, and a probability that the patient will be discharged two or more days from the specific date.

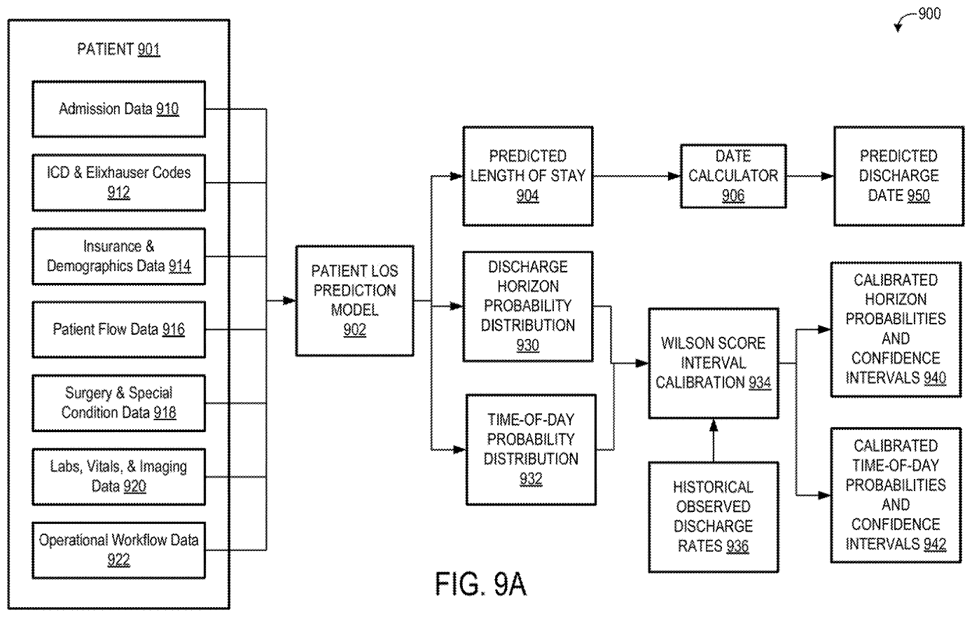

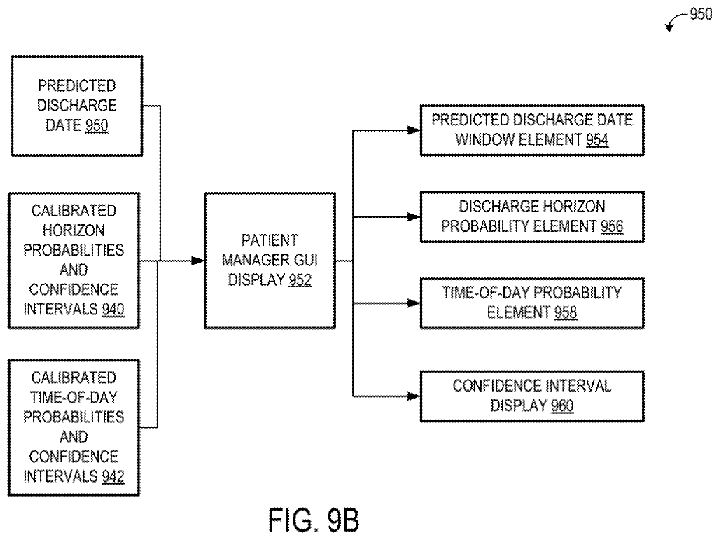

Discharge probability module 209 may further generate a time-of-day discharge probability distribution indicating a probability that the patient will be discharged within a first time window, such as a morning time window (e.g., 12 AM to 11 AM), a probability that the patient will be discharged within a second time window, such as an afternoon time window (e.g., 12 PM to 6 PM), and a probability that the patient will be discharged within a third time window, such as an evening time window (e.g., 7 PM to 11 PM). In various embodiments, discharge probability module 209 may be implemented as one or more output branches of the patient LOS prediction model (e.g., patient LOS prediction model 102 of FIG. 1), where the output branches share one or more layers of the neural network with the LOS prediction output and include separate layers configured to output the discharge horizon probability distribution and the time-of-day discharge probability distribution. In some embodiments, the separate layers may include softmax layers that output probability values summing to one across the categories of each distribution. Discharge probability module 209 may generate the discharge horizon probability distribution and the time-of-day discharge probability distribution simultaneously with the LOS prediction during a single forward pass of a patient data vector through the neural network, as described in greater detail below in reference to FIGS. 9A-14B. The discharge horizon probability distribution and the time-of-day discharge probability distribution generated by discharge probability module 209 may be provided to confidence calibration module 214 for calibration prior to display in a user interface of patient discharge date prediction system 220. Patient discharge date prediction system 220 may include a training module 210, which may comprise instructions for training one or more of the neural networks stored in neural network module 208. Training module 210 may include instructions that, when executed by processor(s) 204, cause patient discharge date prediction system 220 to conduct one or more of the steps of a method for generating a training data set and training a patient LOS prediction model (e.g., the LOS prediction model 102 of FIG. 1), as described in more detail below in reference to FIG. 8. In some embodiments, training module 210 may include instructions for implementing one or more gradient descent algorithms, applying one or more loss functions, and/or training routines, for use in adjusting parameters of one or more neural networks of neural network module 208. Training module 210 may include training datasets for the one or more neural networks of neural network module 208.

Patient discharge date prediction system 220 may include an inference module 212. Inference module 212 may include instructions for deploying a trained patient discharge window prediction model, for example, to predict a discharge window of a patient, as described in FIG. 7. In particular, inference module 212 may include instructions that, when executed by processor 204, cause patient discharge date prediction system 220 to conduct one or more of the steps of the method 700, as described in further detail below.

Patient discharge date prediction system 220 may include a confidence calibration module 214. Confidence calibration module 214 may include instructions that, when executed by processor(s) 204, cause patient discharge date prediction system 220 to apply Wilson score interval calibration to the discharge horizon probability distribution and the time-of-day discharge probability distribution generated by discharge probability module 209. Confidence calibration module 214 may store historical observed discharge rates computed from historical patient data of the healthcare facility, where the historical observed discharge rates represent actual discharge rates observed for patients binned into predicted probability bands during a calibration phase. During inference, confidence calibration module 214 may receive a predicted probability from discharge probability module 209, identify a predicted probability band corresponding to the predicted probability, and retrieve a stored Wilson score interval associated with the identified predicted probability band. The Wilson score interval may include a lower confidence bound and an upper confidence bound around an observed discharge rate for the identified predicted probability band. Confidence calibration module 214 may output calibrated horizon probabilities and confidence intervals (see FIG. 9A) and calibrated time-of-day probabilities and confidence intervals for display in a user interface of patient discharge date prediction system 220. In this way, confidence calibration module 214 may provide care providers with not only a point estimate of discharge probability but also a statistically validated range indicating how reliable the predicted probability has been historically for patients in a similar predicted probability band. The calibration performed by confidence calibration module 214 may be updated periodically as additional historical discharge data becomes available, to maintain accuracy of the confidence intervals over time.

Thus, patient discharge date prediction system 220 may generate predictions of when patients of a healthcare facility may be discharged. In some embodiments, patient discharge date prediction system 220 may generate predictions periodically or regularly at scheduled times. For example, patient discharge date prediction system 220 may generate predictions at several scheduled times during a day. In other embodiments, Patient discharge date prediction system 220 may generate predictions based on an availability of new patient data. For example, if no new patient data is available, patient discharge date prediction system 220 may not generate the predictions, and in response to new patient data becoming available, patient discharge date prediction system 220 may generate (and display) the predictions. In this way, patient discharge date prediction system 220 may perform the task of retrieving patient data relating to the discharge window predictions from EHR database 222 and computer systems and databases 230, and analyzing the retrieved data for the care provider, thereby advantageously reducing an amount of time spent by the care provider finding and analyzing data to determine when a patient may be discharged. Further, the information may be retrieved in background processing to increase computational efficiency.

While not specifically shown in FIG. 2, additional devices described herein (e.g., display device 201) may likewise include user input devices, memory, processors, and communication modules/interfaces similar to communication module 207, non-transitory memory 206, and processor(s) 204 described above, and thus the description of communication module 207, non-transitory memory 206, and processor(s) 204 likewise applies to the other devices described herein. As an example, display device 201 may store user interface templates in memory that include placeholders for relevant information stored on patient discharge date prediction system 220 or received via patient discharge date prediction system 220, which a user of display device 201 may configure. The discharge window predictions and relevant patient information may be retrieved from patient discharge date prediction system 220 and inserted in the placeholders. The user input devices may include keyboards, mice, touch screens, microphones, or other suitable devices.

Turning to FIG. 3, an embodiment of a graphical UI 300 is shown of a patient management application including a patient discharge date prediction system. The patient management application may be a non-limiting embodiment of patient management system 200 of FIG. 2. UI 300 may be a non-limiting example of UI 205 of patient discharge date prediction system 220 of FIG. 2. UI 300 may be displayed on a display device (e.g., display device 201) of the patient management application during operation in a healthcare facility. Specifically, UI 300 may be displayed to a care provider of the healthcare facility when the care provider uses the patient discharge date prediction system to generate discharge window predictions for patients of the healthcare facility, as described in greater detail below in reference to FIGS. 4-8. The discharge window predictions may be used to determine when one or more patients may be discharged and/or to assess an availability of resources (e.g., beds) of the healthcare facility. UI 300 may include a plurality of different individual discharge window predictions for a respective plurality of patients, which may be displayed in rows of UI 300.

UI 300 may be displayed in different alternative views. In some embodiments, UI 300 may be displayed either in a rounding (e.g., enhanced) view, where information is displayed in a fixed format that is not customizable by the user, or in a condensed or command center view, where less information is displayed in a variable format that is customizable by the user. The user may customize the condensed view by hiding one or more columns, rows, sections, or elements of the enhanced view. For example, a first user may view UI 300 in a first condensed view that is customized to hide a first portion of patient data displayed in UI 300, in order to view more rows (e.g., patients) in a single screen. A second user may view UI 300 in a second condensed view that is customized to hide a column of UI 300, in order to more easily view data displayed in a different column. A third user may view UI 300 in a third, enhanced view, in order to view a comprehensive set of data. Each of the first user, the second user, and the third user may select a desired view and/or switch between desired views by selecting one or more controls of UI 300, such as a rounding view toggle switch 302 or a command center view toggle switch 304.

In some embodiments, when UI 300 is displayed, a display panel 315 may automatically generate and show discharge window predictions for patients of a selected medical facility. In other embodiments, display panel 315 may not automatically generate and show the discharge window predictions, and the user may initiate display of the discharge window predictions by selecting a different control element, such as a discharge forecast button 308.

In some embodiments, one or more filters may be applied to UI 300 to filter patient data shown in UI 300. The one or more filters may be selected, for example, via controls accessible via a settings button 309. A summary of one or more filters applied may be displayed in filter bar 310.

As one example, in response to a situation where numerous patients are afflicted with a particular condition, a physician may wish to view discharge window predictions for patients across all hospital units who suffer from the particular condition, in order to determine how many patients may be eligible to discharge free up beds at the hospital units. Using the one or more filters, the user may enter in the particular condition, and select a “display discharge window predictions” button to view the discharge window predictions.

In the embodiment shown in FIG. 3, the discharge window predictions may be displayed in rows of UI 300, where each row corresponds to a patient of the selected hospital unit. The discharge window predictions and other information may be displayed in columns of UI 300. In other embodiments, a layout of the information may be different. For example, in other embodiments, each patient of the selected hospital unit may be displayed in a separate column, and the discharge window predictions and other information may be displayed in rows of UI 300. It should be appreciated that the layout and juxtaposition of the elements of UI 300 may vary in different embodiments, and the elements may appear in different visual configurations without departing from the scope of this disclosure. Additionally, not all of the elements shown in UI 300 may be included in an embodiment, and some embodiments may include a greater or lesser number of elements.

Further, the elements of UI 300 may be controls (e.g., buttons) that may be selected by a user via a user input device (e.g., a mouse, a touchscreen, etc.). When the user selects the a visual element, additional information relevant to the element may be displayed, for example, in a pop-up display panel or on a different screen of UI 300. Further, in some examples, different information may be displayed when the user enters different user input. In such examples, a first pop-up display panel may be displayed when the user selects the element with a first type of user input, such as a hover (e.g., where the user positions a cursor or other user input locator over the element but does not enter further user input). A second pop-up display panel may be displayed when the user selects the element with a second type of user input, such as selecting the element with one or more clicks or touches.

UI 300 may include a patient info column 316, which may show identifying and general information of a patient. For each row of UI 300, a patient data panel 332 that includes patient information may be displayed for a corresponding patient in the patient info column 316. Patient data panel 332 may include a patient name (or abbreviated name) 324. A location code 326 may also be included in the patient data panel 332, which may indicate a current location of the patient in the hospital or hospital system (e.g., the ICU to which the patient has been assigned and/or is currently registered). One of more patient location icons 328 may also be displayed, which may indicate, for example, patient location information such as a current unit in which the patient is being taken care of, as well as historical predictions along a past patient pathway. Patient data panel 332 may also include other general information, such as, for example, an identification number of the patient, an age and/or date of birth of the patient, an attending physician or nurse of the patient, patient insurance information, a diagnosis of the patient, and the like. Further, some aspects of patient information may be visualized via one or more icons 330.

In some embodiments, a care communication button 334 may be included in patient data panel 332, which when selected may generate an alert that may be sent to other care providers and/or saved as part of the patient's EHR to indicate that an ordered or commanded patient task has yet to be initiated or completed. However, in some examples, care communication button 334 may be omitted or may trigger other types of communication or alerts.

UI 300 may include a dates column 318, where dates column 318 includes a dates panel 336 for each patient displayed. Dates panel 336 may show a timeline of events relating to changes in a status of each patient during a time spent by the patient at the healthcare facility, including dates and times of the events. In some embodiments, the timeline may begin at a time of admission of a patient, which may be indicated via an admit graphical element 360. Dates panel 336 may also include an expected discharge date (EDD) element 364, which may indicate a projected discharge data based on historical data collected from a plurality of patients with a same health condition as the patient. In other embodiments, a mean length of stay for patients with the same condition as the patient (e.g., a geometric mean length of stay, GMLOS) may be included. Additionally, as described in greater detail below, the patient discharge date prediction system may predict a discharge date 366 for one or more patients based on an output of a patient discharge date prediction model. In various embodiments, dates panel 336 may include a predicted discharge date window element 362, where the predicted discharge date window is based on the predicted discharge date. For example, the predicted discharge date window may be a date range including the predicted discharge date. Additionally or alternatively, the predicted discharge date window may be expressed relative to a current day, such as “In 1-2 days”, “In 1 week”, or similar description.

Additionally, a length of the discharge window (e.g., a number of days of the date range) may be based on a desired confidence level, where the desired confidence level reflects a level of confidence that the patient may be discharged within the discharge window. For example, if the desired confidence level is high (e.g., 80%), the length of the discharge window may be extended to include a wider range of dates, to achieve the desired level of confidence that the patient may be discharged within the discharge window. Alternatively, if the desired confidence level is lower (e.g., 50%), the length of the discharge window may be reduced to include a narrower range of dates. If the desired confidence level is very low (e.g., 10%), the predicted discharge date window may be the predicted discharge date.

In various embodiments, the desired confidence level may be configured in the patient discharge date prediction system by a user, or an administrator of the healthcare facility. For example, in some embodiments, the user may select the desired confidence level via a menu of UI 300, such as by selecting a setting from a menu displayed as a result of the user selecting the settings button 309. In other embodiments, the administrator may pre-configure to desired confidence level based on administrative guidelines of the healthcare facility. The confidence level may be displayed in or next to the predicted discharge date window element 362, so that the user may see the confidence level used to generate the discharge window prediction from the predicted discharge date.

UI 300 may include an alerts column 322, which may show one or more patient alerts. The one or more alert elements 342 may be included in a row of UI 300 corresponding to a patient. The one or more alert elements 342 may include patient alerts generated by one or more EHR systems or other computer systems of a healthcare organization of which the hospital unit is a part (e.g., EHR database 222 and/or computer systems and databases 230 of FIG. 2). For example, an EHR system may generate a flag for a patient indicating that the patient is overdue for a test or examination, that certain records or paperwork are missing, that a patient is scheduled for a procedure but has lab results to be consulted prior to performing the procedure, or other suitable alert. The one or more alert elements 342 may also include one or more pending discharge milestones, representing tasks to be carried out or completed prior to discharging the patient, pending reports or results, operational logistics involved in transferring the patient, and/or other reminders, warnings, or additional information. In some embodiments, the one or more alert elements 342 may be controls that may be selected. When an alert element 342 is selected, additional information regarding the alert may be displayed via an additional (e.g., pop-up) display panel, or an external system (such as an EHR system) may be launched to allow a user to view the additional information.

In some embodiments, the patient discharge date prediction system may run regularly or periodically, and the predicted patient discharge window and/or patient data may be periodically updated automatically by the patient discharge date prediction system. In other embodiments, the patient discharge date prediction system may be launched by a care provider when the care provider wishes to determine if one or more patients of an ICU may be discharged. For example, a care provider may start the patient discharge date prediction system and view predicted discharge date window elements 362 of a plurality of patients at a first time. At the first time, the care provider may see that no patients are close to a predicted discharge date window. The care provider may leave the patient manager to attend to patients, and return later to the patient manager, to view the predicted discharge date window elements 362 of the plurality of patients at a second time. In between the first time and the second time, elements of patient data and/or discharge window predictions may change, for example, due to more recent data being recorded in one or more EHR systems. As a result of the more recent data being recorded, at the second time, the care provider may see that a patient that was previously not close to a predicted discharge date window is now close to the predicted discharge date window. As a result of seeing that the patient is now close to the predicted discharge date window, the care provider may initiate one or more tasks in preparation for discharging the patient. For example, the tasks may include initiating paperwork, arranging for transportation, preparing to place a new patient, preparing the patient for discharging, and managing resources and tasks such as medical equipment, medication, home care resources, and others depending on a lead time for management. Thus, the patient discharge date prediction system may serve to provide updated data that may aid the care provider in managing patients of the healthcare facility.

In some embodiments, if a current date falls within the predicted discharge date window, the patient discharge date prediction system may generate an alert at the predicted discharge date window element 362 indicating to the care provider that a discharge of a patient may be pending or approaching. For example, a level of illumination of the predicted discharge date window element 362 may be increased, or a color of the predicted discharge date window element 362 may be changed, or a different visual indication of the pending or approaching discharge may be provided. Further, in some embodiments, if the current date falls within the predicted discharge date window, the patient discharge date prediction system may generate one or more notifications to one or more care providers of the patient. For example, a message may be sent to the one or more care providers indicating one or more administrative tasks to be performed prior to discharging the patient, such as paperwork to be filled out, transportation arrangements to be made, family members to be contacted, and the like.

In embodiments implementing the discharge horizon probability distribution and the time-of-day discharge probability distribution described below in reference to FIGS. 9A through 14B, the patient discharge date prediction system may automatically initiate one or more discharge preparation workflows in response to determining that a probability of the discharge horizon probability distribution indicating that the patient will be discharged on a current date exceeds a predetermined threshold probability. For example, the predetermined threshold probability may be 60%, 70%, 80%, or another value configured by an administrator of the healthcare facility or selected by a care provider via a settings menu of UI 300. In response to determining that the same-day discharge probability exceeds the predetermined threshold probability, the patient discharge date prediction system may automatically initiate one or more discharge preparation workflows for the patient. The one or more discharge preparation workflows may include generating a notification to one or more care providers of the patient indicating one or more administrative tasks to be performed prior to discharging the patient, arranging transportation for the patient, initiating paperwork associated with discharging the patient, and generating a message to coordinate staffing, pharmacy, or post-acute placement services based on the time-of-day discharge probability distribution. For example, if the discharge horizon probability distribution indicates a 75% probability that the patient will be discharged on a current date, and the predetermined threshold probability is 70%, the patient discharge date prediction system may generate a notification to a case manager of the patient indicating that discharge preparation tasks should be initiated, and may further generate a message to a transport coordinator indicating that patient transport should be scheduled for the patient.

Further, in response to determining that a probability of the time-of-day discharge probability distribution indicating that the patient will be discharged within a particular time window exceeds a second predetermined threshold probability, the patient discharge date prediction system may automatically initiate time-specific discharge coordination workflows. The second predetermined threshold probability may be the same as or different from the predetermined threshold probability used for the discharge horizon probability distribution. The time-specific discharge coordination workflows may include scheduling patient transport within the particular time window, notifying bed management of anticipated bed availability within the particular time window, and coordinating pharmacy medication reconciliation for completion prior to the particular time window. For example, if the time-of-day discharge probability distribution indicates a 72% probability that the patient will be discharged within a morning time window (e.g., 12 AM to 11 AM), and the second predetermined threshold probability is 65%, the patient discharge date prediction system may generate a message to a transport coordinator indicating that patient transport should be scheduled within the morning time window, may generate a notification to bed management indicating that the patient's bed is anticipated to become available within the morning time window, and may generate a message to a pharmacy indicating that medication reconciliation for the patient should be completed prior to the morning time window. In this way, the patient discharge date prediction system may use the time-of-day discharge probability distribution to coordinate time-sensitive discharge logistics in advance of the predicted discharge time window, thereby reducing last-minute coordination delays that may otherwise delay the actual discharge of the patient.

In various embodiments, the predetermined threshold probability and the second predetermined threshold probability may be configurable by an administrator of the healthcare facility or by a care provider via a settings menu of UI 300 (e.g., via settings button 309). Different threshold probabilities may be configured for different types of discharge preparation workflows. For example, a first threshold probability of 60% may be configured for initiating paperwork and notification workflows, while a second, higher threshold probability of 80% may be configured for scheduling transport and notifying bed management, to reduce a likelihood of prematurely scheduling resources for patients whose discharge may not occur as predicted. In other embodiments, a single threshold probability may be used for all discharge preparation workflows.

Thus, while patient data relevant to patient discharges is updated asynchronously in one or more systems and/or databases coupled to the patient discharge date prediction system, a predicted discharge date window of a patient based on a most recent version of the patient's data may be displayed in UI 300. In this way, the patient discharge date prediction system may provide an efficient and user-friendly way to monitor patient discharge information via a single interface, without having to repeatedly access various EHR systems to ensure accuracy of the patient data and/or while the various EHR systems are in an unlaunched state (e.g., not consuming computing and/or network resources of the healthcare system). Additionally, in some embodiments, a “last update date/time” may be included in the predicted discharge date window element 362, which may indicate to the user the last time that the discharge window prediction was updated.

As an example of how UI 300 may be used, a care provider may oversee an unit of a hospital with 10 patients, where all beds of the unit are occupied. The care provider may receive a request from an administration of the hospital to admit another patient. In order to determine whether the patient may be admitted to the unit, the care provider may wish to determine whether one of the 10 current patients may be discharged, to make a bed of the unit available.

The care provider may start a software application (e.g., the patient management application) running the patient discharge date prediction system in one of the ways described above, upon which UI 300 may appear on a display device. The care provider may select a scope of the patient date discharge prediction system (e.g., the unit of the hospital). The patient discharge date prediction system may retrieve a list of patients corresponding to the unit from one or more EHRs and/or hospital computer systems and databases, and generate a plurality of discharge window predictions for the patients on the list. When discharge window predictions have been generated by the patient discharge date prediction system for all of the patients on the list of patients, the discharge window predictions may be outputted to UI 300 for display. Predefined visual elements/controls that correspond to the discharge window predictions (e.g., predicted discharge date window element 362) may be selected and inserted into a template of UI 300 along with corresponding patient identifying data. Additionally, patient alert, discharge milestone, and other elements may be generated (based, for example, on one or more algorithms included in the software application) and displayed in relevant patient rows of UI 300.

When UI 300 displays the discharge window predictions generated by the patient discharge date prediction system, the care provider may see that a patient of the 10 patients of the unit has a predicted discharge date window that includes a current date, indicating that a discharge of the patient is predicted to be imminent. In some embodiments, the 10 patients of the unit may be displayed in UI 300 in a descending order according to a proximity of each patient to a predicted discharge date window of the patient. For example, the patient with the predicted discharge date window that includes the current date may be displayed in a first (e.g., top) row of UI 300; a second patient with a predicted discharge date window starting closest to the current date may be displayed in a second row of UI 300; a third patient with a predicted discharge date window next closest to the current date may be displayed in a third row of UI 300; and so on. In other embodiments, the 10 patients may not be displayed in the descending order, and the predicted discharge date window element 362 for the patient with the predicted discharge date window that includes the current date may be highlighted by a change of color, brightness, a border, and/or an icon, whereby the care provider may easily identify the patient as being predicted to be imminently discharged.

In some embodiments, the care provider may select the predicted discharge date window element 362 of the patient predicted to be imminently discharged, to pop up an additional display panel with more information about the predicted discharge date window. In some embodiments, the care provider may review the information, and confirm the predicted discharge date window by selecting a “confirm” button on the additional display panel. When the discharge window prediction is confirmed, the care provider may be prompted to initiate one or more actions related to discharging the patient, and/or automated workflows may be created based on discharge predictions to prepare care providers and initiate messages for managing resources and discharge tasks.

Additionally or alternatively, via UI 300, the care provider may view one or more alerts generated for the patient, and initiate any tasks that may be entailed by a discharge. As the provider completes a task or meets a milestone relevant to the discharge, the provider may dismiss one or more of the one or more alerts, for example, by selecting a control in a pop-up display panel triggered by selecting an alert. Before closing the application, the care provider may review the predicted discharge date windows of other patients on the list to see when the other patients are predicted to be discharged. The care provider may see that a second patient may be ready to be discharged, and the care provider may initiate a discharge process for the second patient. Alternatively, the care provider may determine that the predicted discharge date window for the second patient is not accurate, for example, based on current patient data available to the care provider but not yet updated in the EHR. If the care provider determines that the predicted discharge date window for the second patient is not accurate, the care provider may update the predicted discharge date window. For example, the care provider may upload additional patient information to the EHR that causes the predicted discharge date window to change, whereby the predicted discharge date window element 362 for the second patient may be updated in UI 300. Additionally or alternatively, the care provider may select discharge window element 362 of the second patient, and insert a comment which may be viewed by one or more other care providers or administrators of the hospital.

Once the patient is discharged, the patient discharge date prediction system may receive an updated location of the patient. Once the patient location is updated, the predicted discharge date window element 362 may be removed from UI 300 for the hospital unit.

Referring now to FIG. 4, a patient care timeline 400 is shown, indicating various periods during which data relevant to predicting a discharge date of a patient of a hospital network is collected. Patient care timeline 400 may be divided into three segments: a patient history segment 402, a current visit segment 404, and a care progression segment 406. Patient data from each of the three segments may include different factors taken into consideration by a patient discharge date prediction system, such as the patient discharge date prediction system 220 of FIG. 2.

Patient history segment 402 may include patient data collected from a beginning of the patient's medical history (e.g., where a time T of patient care timeline 400 is 0), and may extend until a time T=1 of the patient's admission, indicated by a dashed line 410. Patient history segment 402 may include patient data from previous hospital visits, prior conditions suffered by the patient, previous diagnoses and treatments performed within the hospital network, or in other hospital networks, and similar historical healthcare information collected on the patient. Patient data collected during patient history segment 402 may include length of stay data relating to the previous hospital visits, which may be used by an LOS prediction model (e.g., LOS prediction model 102 of FIG. 1) to predict a length of stay of the patient during a current visit at a hospital. For example, previous hospital visit data of the patient may indicate that the patient typically recovers quickly, or that the patient often takes longer than usual to recover.

Current visit segment 404 may include patient data collected from a beginning of the patient's current visit (e.g., at the time T=1 indicated by dashed line 410), and may extend until a time T=2, indicated by a dashed line 412. Current visit segment 404 may include patient data collected initially when the patient is admitted to the hospital. For example, current visit segment 404 may include a primary diagnosis for which the patient is admitted. Current visit segment 404 may include a care pathway, indicating an expected trajectory of the patient through various procedures and treatments. Current visit segment 404 may include data on surgical or other procedures performed on the patient during the current hospital visit. Additionally, current visit segment 404 may include descriptive information of the patient that may influence an amount of time that the patient may remain at the hospital. For example, patient data collected during current visit segment 404 may include a body mass index (BMI), where a larger BMI may indicate a longer recovery time that a smaller BMI. The patient data may include a current age of the patient, where an older patient may have a longer recovery time that a younger patient. The patient data may include other, less serious health conditions and/or health conditions unrelated to the primary diagnosis that the patient may be experiencing. For example, the patient data may include a psychological or emotional state of the patient, or a chronic condition that may complicate treatment of a primary condition of the patient.

Care progression segment 406 may include patient data collected on an ongoing basis over the course of the patient's treatment, starting from the time T=2 indicated by dashed line 412, and extending until a time T=3, indicated by a dashed line 414. Time T=3 may represent an earliest time at which the patient may be discharged from the hospital. Care progression segment 406 may include a current diagnosis of the patient and a current location of the patient within the hospital network. Care progression segment 406 may include patient monitoring information, such as vital sign information collected from one or more patient monitors arranged at or on the patient. Care progression segment 406 may include lab data, or other test results carried out on the patient during treatment. Care progression segment 406 may include a duration of care (e.g., how long a patient has been receiving care for a specific condition).

Patient data from patient history segment 402, current visit segment 404, and care progression segment 406 of patient care timeline 400 may be used as input into the LOS prediction model to predict the patient length of stay at the hospital. Based on the predicted LOS, a predicted discharge date 450 may be generated, where predicted discharge date 450 may be a date after time T=3 (e.g., after an estimated end of care progression segment 406). Further, predicted discharge date 450 may be used to generate a predicted discharge date window 418, which in FIG. 4 extends from time T=3 to a time T=4, indicated by a dashed line 416. Predicted discharge date window 418 may be a date range within which a probability of a discharge of the patient may exceed a pre-configured threshold probability. For example, the pre-configured threshold probability may be 80%, where predicted discharge date window 418 may be a range of dates within which a probability of the patient being discharged exceeds 80%. In other words, the pre-configured threshold probability may reflect a level of confidence that predicted discharge date window 418 is 80% accurate. Thus, a long predicted discharge date window 418 may correspond to a high level of confidence, and a short predicted discharge date window 418 may correspond to a low level of confidence.

In addition to the predicted discharge date window 418, the patient discharge date prediction system may generate a discharge horizon probability distribution over possible discharge times. For example, the discharge horizon probability distribution may indicate relative probabilities that the patient will be discharged on a specified date (e.g., a current date), the day after the specified date, or two or more days from the specified date, and a time-of-day discharge probability distribution indicating relative probabilities that the patient will be discharged within a first time window on a discharge day, a second time window on the discharge day, or a third time window on the discharge day. As time T progresses through care progression segment 406, the discharge horizon probability distribution and the time-of-day discharge probability distribution may become increasingly precise as additional patient data becomes available.

FIG. 5A shows a patient data schematic 500 where different types of patient data of a patient are stacked in lanes of a timeline, to visually indicate when the different types of patient data are collected. The patient data shown in patient data schematic 500 may be the same as the patient data collected over patient care timeline 400 of FIG. 4. The timeline extends from a time T=0 to a time T=19, where the times T may indicate consecutive dates during the patient's stay at a hospital. For example, a time T=1 may indicate a first day on which the patient was admitted to the hospital; a time T=2 may indicate a second day after the patient was admitted to the hospital; and so on.

Patient data schematic 500 includes six lanes: a treatment lane 501, in which treatment data collected at different times as shown; a diagnosis lane 509, in which diagnosis data collected at different times as shown; an imaging lane 519, in which imaging data collected at different times as shown; a surgeries lane 521, in which surgery data collected at different times as shown; a labs lane 525, in which lab data collected at different times as shown; and a vitals lane 529, in which vital sign data collected at different times as shown. It should be appreciated that the types of data shown in FIG. 5A are for illustrative purposes, and in other embodiments a greater or lesser number of lanes, or different types of patient data, may be included without departing from the scope of this disclosure.

Treatment lane 501 shows a first set of treatments 502 performed on the patient, where the first set of treatments 502 are of a treatment type 503. The first set of treatments 502 includes four treatments, where the four treatments are performed at times T=1, 2, 3, and 4. Treatment lane 501 shows a second set of treatments 504 performed on the patient, where the second set of treatments 504 are of a treatment type 505. The second set of treatments 504 includes two treatments, where the two treatments are performed at times T=5 and 6. Treatment lane 501 shows a third set of treatments 506 performed on the patient, where the third set of treatments 506 are of a treatment type 507. The third set of treatments 507 includes three treatments, where the three treatments are performed at times T=7, 8, and 9.

The patient data depicted in diagnosis lane 509 includes various diagnoses made at different times between a time T=1 and T=9. For example, at a time T=2 (e.g., on a second day at the hospital), three diagnoses were made: a first diagnosis 510, a second diagnosis 511, and a third diagnosis 512. Similarly, patient data schematic 500 shows in imaging lane 519 that a first imaging study 514 and a second imaging study 515 were performed at time T=2, and a third imaging study 516 and a fourth imaging study 517 were performed at time T=6. The patient data depicted in surgery lane 521 includes a first surgery 518 performed at time T=3, and a second surgery 520 performed at time T=7. Patient data schematic 500 shows in lab lane 525 that at time T=3, a first lab result 522 of a first lab test, a second lab result 523 of a second lab test, and a third lab result 524 of a third lab test were recorded. In the vitals lane 529 of patient data schematic 500, it can be seen that at time T=9, readings of a first vital sign 526, a second vital sign 527, and a third vital sign 528 were performed.

The patient data depicted in patient data schematic 500 may be used to predict an LOS of the patient, which in turn may be used to predict a discharge date window within which the patient may be discharged. To predict the LOS of the patient, the patient data included in patient data schematic 500 may be inputted into an LOS prediction model (e.g., the LOS prediction model 102 of FIG. 1), where an output of the LOS prediction model is the predicted LOS of the patient. In various embodiments, the patient data may be inputted into the LOS prediction model as a data vector including an encoding of the patient data, as shown in FIG. 5B.

FIG. 5B shows an exemplary data vector diagram 550, including a data vector 552 comprising encoded patient data, which may be used as input into the LOS prediction model. Data vector 552 may be divided into sections, where each section includes patient data corresponding to a patient data category. In the embodiment shown in FIG. 5B, the patient data categories correspond to the lanes of the timeline depicted in patient data schematic 500 of FIG. 5A. Thus, a first section 554 may correspond to patient data of treatment lane 501. A second section 556 may correspond to patient data of diagnosis lane 509. A third section 558 may correspond to patient data of imaging lane 519. A fourth section 560 may correspond to patient data of surgeries lane 521. A fifth section 562 may correspond to patient data of labs lane 525. A sixth section 564 may correspond to patient data of vitals lane 529. In other embodiments, the patient data categories may correspond to different types of patient data, where there may be a greater or lesser number of different types of patient data than the patient data categories shown in FIG. 5B. In different embodiments, the patient data may be categorized differently. For example, the patient data may be first classified into clinical and nonclinical data, and the clinical data and nonclinical data may both be further subcategorized.

Each section of data vector 552 may include a plurality of consecutive values 566, where the plurality of consecutive values 566 represent an encoding of patient data included in the patient data category corresponding to the section. A number of the plurality of consecutive values 566 may be based on an amount of patient data of the corresponding patient data category. For example, third section 558 may include less consecutive values 566 than fifth section 562, as a result the amount of surgery data of the patient being less than an amount of lab data of the patient.

The plurality of consecutive values 566 included in each section of data vector 552 may be encoded using various different encodings. For example, the consecutive values 566 may include binary values, where the patient data is encoded as a plurality of 0s and 1s, as shown in the third section 558 corresponding to imaging data (e.g., the imaging data of imaging lane 519). In other words, the binary string “0 1 1 0 0” may represent first imaging study 514 and second imaging study 515 performed at time T=2, and third imaging study 516 and a fourth imaging study 517 performed at time T=6, of FIG. 5A. In other embodiments, different types of encodings may be used. For example, a unique number may be assigned to each data point of a patient data category, and data vector 552 may comprise a set of all numbers corresponding to patient data of the patient. In some embodiments, data vector 552 may include additional sections corresponding to additional data categories, such as operational workflow data, as described in greater detail below in reference to FIG. 9A.

In various embodiments, the LOS prediction model may be a neural network model, such as a convolutional neural network (CNN), and data vector 552 may be inputted into an input layer of the CNN.

Referring now to FIG. 6A, an example of an LOS prediction model training system 600 is shown, where the LOS prediction model is a neural network model including a CNN. LOS prediction model training system 600 may be implemented by a patient discharge date prediction system, such as patient discharge date prediction system 100 of FIG. 1, to train the CNN to predict a length of stay of a patient based on patient data of the patient. In an embodiment, LOS prediction model training system 600 includes a patient LOS prediction model 602, to be trained, which may be part of a neural network module 604 of the patient discharge date prediction system (e.g., neural network module 208 of FIG. 2).

The patient LOS prediction model may be trained on a training dataset, which may be stored in a training module 606 (e.g., training module 210 of FIG. 2). The training dataset may comprise a plurality of training pairs 608. Each training pair 608 may include input data, and ground truth target data.

In an embodiment, the input data of each training pair 608 may be a data vector 610, where data vector 610 is a vector of values encoding patient data of a historical patient (e.g., data vector 552 of FIG. 5B). The ground truth target data may be an actual LOS 612 of the historical patient, which may be the length of stay of the patient corresponding to data vector 610. Data vector 610 and actual LOS 612 of training pair 608 may be obtained from historical patient data 614. Historical patient data 614 may be archived and de-identified patient data stored, for example, in one or more databases of a hospital network (e.g., computer systems and databases 230 of FIG. 2).

In various embodiments, data vector 610 and actual LOS 612 may be generated from historical patient data 614 by a dataset generator 616. In one embodiment, dataset generator 616 may organize and/or structure historical patient data 614 in the stacked, timeline approach as depicted in patient data schematic 500 of FIG. 5A, to generate data vector 610 in the manner described in reference to FIG. 5B. The data may be stacked based on data categories, or the data may be stacked temporally.

Once the training pairs 608 have been generated, a portion of the training pairs 608 may be assigned to a test dataset, as test pairs 618. The test dataset may be used to prevent overfitting, whereby patient LOS prediction model 602 may learn to map features specific to samples of the training set that are not present in the test set. In some embodiments, the training pairs 608 may be randomly assigned to either the training dataset or the test dataset in a pre-established proportion. For example, 90% of the training pairs 608 generated may be assigned to the training dataset, and 10% of the training pairs 608 generated may be assigned to the test dataset. In other embodiments, different proportions of training pairs 608 may be assigned to the training dataset and the test dataset. As a non-limiting example, the number of training pairs 608 used may be 10,000, and the number of test pairs 618 used may be 1000. It should be appreciated that the examples provided herein are for illustrative purposes, and the training pairs 608 may be assigned to the training dataset or the test dataset via a different procedure and/or in a different proportion without departing from the scope of this disclosure. In some embodiments, a training data set, test data set, and validation data set may be generated, for example, based on randomly selected patients clustered based on various factors.

LOS prediction model training system 600 may be implemented to train patient LOS prediction model 602 to learn to predict the actual LOS 612 of the patient. Patient LOS prediction model 602 may be configured to receive training pairs 608 from the training module 606, where data vector 610 and the corresponding actual LOS 612 are inputted into patient LOS prediction model 602. Patient LOS prediction model 602 may output a predicted LOS of the patient based on the data vector 610. Patient LOS prediction model 602 may then iteratively adjust one or more parameters of patient LOS prediction model 602 in order to minimize a loss function (e.g., based on a difference between the predicted LOS and actual LOS 612), until an error rate decreases below a first threshold error rate.

LOS prediction model training system 600 may include a validator 620 that validates a performance of patient LOS prediction model 602. Validator 620 may take as input a trained or partially trained patient LOS prediction model 602 and a test dataset of test pairs 618. If the error rate of the trained or partially trained patient LOS prediction model 602 on the test dataset of test pairs 618 decreases below a second threshold error rate, the performance of the trained or partially trained patient LOS prediction model 602 may be validated, whereby a training stage of the trained or partially trained patient LOS prediction model 602 may end.

For example, a partially trained patient LOS prediction model 602 of LOS prediction model training system may be validated with a test dataset of 50 test pairs 618, where each of the 50 test pairs 618 comprises a data vector 610 of a historical patient and an actual LOS 612 of the historical patient. Validator 620 may feed data vector 610 and actual patient LOS 612 into the partially trained patient LOS prediction model 602 and receive a predicted LOS as an output. Validator 620 may then compare the predicted LOS with the actual LOS 612. If an error between the predicted LOS and the actual LOS 612 is below the threshold error, the partially trained patient LOS prediction model 602 may be validated. After validation, a trained patient LOS prediction model 622 may be used to predict an LOS of a new patient based on patient data of the new patient, as described below in reference to FIG. 6B.

FIG. 6B shows a deployment diagram 650 of trained patient LOS prediction model 622 of FIG. 6A in use during deployment within a patient discharge date prediction system 654, which may be a non-limiting example of patient discharge date prediction system 100 of FIG. 1. Trained patient LOS prediction model 622 may be used to predict a LOS of a current patient of a hospital based on patient data of the patient. Validated patient LOS prediction model 622 may be included within an inference module 652 (e.g., inference module 212 of FIG. 2) of patient discharge date prediction system 654. Inference module 652 may also include instructions for deploying validated patient LOS prediction model 622 to generate a predicted LOS of the patient.

During operation of the patient discharge date prediction system 654, a set of current patients 656 may be selected for generating discharge date predictions. For example, as described above in reference to FIG. 3, the set of current patients 656 may be a group of patients being attended to at a hospital unit selected by a care provider via UI 300. The set of current patients 656 includes a plurality of individual patients 658, where each patient 658 is represented by a data vector 660 (e.g., data vector 552) generated, for example, in the manner described in FIG. 5B.

For each patient 658 of the set of current patients 656, a corresponding data vector 660 may be inputted into validated patient LOS prediction model 622, to output an LOS prediction 662 for the patient 658. For example, a first data vector 660 of a first patient 658 may be inputted into validated patient LOS prediction model 622 to generate a first LOS prediction for the first patient 658; a second data vector 660 of a second patient 658 may be inputted into validated patient LOS prediction model 622 to generate a second LOS prediction for the second patient 658; and so on, until an LOS prediction 662 has been generated for each patient 658 of the set of current patients 656. The patient discharge date prediction system 654 may then generate predicted discharge dates for each patient 658 of the set of current patients 656 based on the LOS predictions 662. Each predicted discharge date of each patient 658 may be used to generate a corresponding predicted discharge date window, based on a confidence level selected by the care provider. The patient discharge date prediction system 654 may then display a predicted discharge date window element indicating the predicted discharge date window in a UI of patient discharge date prediction system 654 (e.g., UI 300).

Referring now to FIG. 7, a flowchart of an exemplary method 700 is shown for training a CNN-based patient LOS prediction model, such as patient LOS prediction model 602 of FIG. 6A, to predict a length of stay of a patient at one or more hospitals of a hospital network. Method 700 may be implemented as part of the LOS prediction model training system 600 of FIG. 6A. In an embodiment, one or more operations of method 700 may be stored in non-transitory memory and executed by a processor, such as the non-transitory memory 206 and processor 204 of patient discharge date prediction system 220 of FIG. 2.

The patient LOS prediction model may be trained using a training dataset comprising sets of input/target training pairs (e.g., training pairs 608). Each training pair of the input/target training pairs may include a data vector of a patient, and an actual LOS (e.g., ground truth) of the patient.

Method 700 begins at operation 702, where method 700 includes receiving historical patient data from one or more databases of the hospital network (e.g., computer systems and databases 230 of FIG. 2).

At 704, method 700 includes dividing the historical patient data into a plurality of groupings. In various embodiments, the groupings may be based on length of stay information in the historical patient data. For example, if the historical patient data includes patients with a length of stay ranging from one day to 90 days, then the 90 days may be divided into nine different 10 day groupings. A first ten-day grouping may include any patients with an LOS between one day and 10 days; a second grouping may include any patients with an LOS between 10 days and 20 days; a third grouping may include patients with an LOS between 20 days and 30 days; and so on. A number of groupings into which the historical patient data is divided may depend on a homogeneity of the historical patient data with respect to length of stay. For example, if the LOS's of patients of the historical patient data fall within a short range, a small number of groupings may be used, and if the LOS is of patients of the historical patient data fall within a wide range, a larger number of groupings may be used. Other factors may additionally be used to determine the number of groupings into which the historical patient data is divided. In some cases, a number of patients within each grouping may be used to determine a feasibility of using the grouping for training/validation.

In other embodiments, the groupings may be based partially or totally on factors other than length of stay. For example, a clustering algorithm, or a machine learning or deep learning algorithm may be used to identify groupings into which the patients cluster based on patient data and/or other data.

At 706, method 700 includes classifying patients according to the groupings. In various embodiments, each patient of the historical patient data may be assigned a grouping depending on the LOS of the patient. For example, a first set of patients having a short LOS may be assigned to a first grouping; a second set of patients having a longer LOS than the first set of patients may be assigned to a second grouping; a third set of patients having a longer LOS than the second set of patients may be assigned to a third grouping; and so on. In embodiments, where the groupings are generated based on the clustering algorithm or machine learning or deep learning algorithm, each patient of the historical patient data may be assigned a grouping based on the patient data used to generate the groupings.

At 708, method 700 includes generating data vectors for each patient of the historical patient data. The data vectors may be the same as or similar to data vector 552 described above in reference to FIG. 5B.

At 710, method 700 includes creating training pairs for each patient, each training pair including a data vector of the patient as input data, and an LOS of the patient as ground truth data. In some embodiments, the training set may be stored in a training module of an image processing system, such as the training module 210 of patient discharge date prediction system 220 of FIG. 2. In some embodiments, the data vectors may be generated by a dataset generator of the training module, such as dataset generator 616 of FIG. 6A.

At 712, method 700 includes training the CNN on each grouping to predict an LOS of each patient of each grouping. During training of the CNN, each data vector of a training pair of a grouping may be inputted into the CNN (e.g., into an input layer of the CNN) to generate a predicted LOS of the patient corresponding to the data vector as an output. For example, patient data (e.g., data vectors) of a first grouping may be inputted into the CNN in a first training step. After training on the first grouping has completed, patient data of a second grouping may be inputted into the CNN in a second training step, and so on.

At 714, training the CNN on each grouping includes propagating input values of the data vector through layers of the network, from the input layer, through one or more hidden layers, until reaching an output layer of the CNN. Propagating input values of the data vector through layers of the network may include performing convolutions and/or pooling operations on the data vector.

For example, in an embodiment, the CNN may have two convolutional layers, a fully connected layer, and an output layer. In some embodiments, pooling layers may be included between a first convolutional layer and a second convolutional layer, and/or between the second convolutional layer and the fully connected layer. Convolutions may be performed on the input values at the first convolutional layer of the CNN, resulting in a first set of feature maps. The first set of feature maps outputted by the first convolutional layer may be pooled at a first pooling layer, resulting in pooled feature maps. A first set of computed values of the pooled feature maps outputted by the first pooling layer may be inputted into the second convolutional layer. Convolutions may be performed on the first set of computed values at the second convolutional layer, resulting in a second set of feature maps. The second set of feature maps outputted by the second convolutional layer may be pooled at a second pooling layer, resulting in a second set of pooled feature maps. A second set of computed values of the second set of pooled feature maps outputted by the second pooling layer may be inputted into a fully connected layer of the CNN, which may generate one or more output values of the CNN at an output layer. In various embodiments, the one or more output values may include an encoding of an LOS of the patient, as described above in reference to FIG. 6A. For example, the output may be a number of days that the patient is predicted to stay at the hospital.

In some embodiments, outputs of the first convolutional layer, the second convolutional layer, and the fully connected layer may be modified by an activation function prior to being inputted into the first pooling layer, the second pooling layer, and the output layer, respectively. For example, the activation function may be a rectified linear activation function (ReLU).

After propagating the input values of the data vector through the layers of the CNN, a plurality of weights and biases at each layer of the CNN may be adjusted via backpropagation. In various embodiments, adjusting the plurality of weights and biases of the CNN may include calculating a difference between the output of the CNN and the actual LOS of the patient inputted into the CNN as a target ground truth value along with the data vector. The plurality of weights and biases of the CNN may be adjusted based on the difference between the output and the target ground truth from a relevant training pair. The difference (or loss), as determined by a loss function, may be backpropagated through the CNN to update weights of the convolutional layers. In some embodiments, backpropagation of the loss may be carried out according to a gradient descent algorithm, wherein a gradient of the loss function (a first derivative, or approximation of the first derivative) is determined for each weight and bias of the CNN. Each weight (and bias) of the CNN may be updated by adding the negative of the product of the gradient determined (or approximated) for the weight (or bias) with a predetermined step size. The backpropagation may comprise computing the gradient one layer at a time, iterating backward from the last layer to the first layer. It will be noted that method 700 may be repeated until the weights and biases of the CNN converge, or the rate of change of the weights and/or biases of the CNN for each iteration of method 700 are below a threshold rate of change.

At 716, training the CNN on each grouping further includes validating and assigning an accuracy rating to the grouping. In order to avoid overfitting, training of the CNN may be periodically interrupted to validate a performance of the CNN on a test set comprising test pairs, such as test pairs 618 of FIG. 6A. In one example, both the training pairs of the training set and the test pairs of the test set may be selected at random from a larger training dataset. In some embodiments, training of the CNN may end when the performance of the CNN on the test training pairs converges (e.g., when an error rate on the test set converges on a minimum value).

In some embodiments, after training the CNN on a grouping, an accuracy rating may be assigned to the grouping. For example, a first grouping including patients with an LOS within a first range may be assigned a first accuracy rating; a second grouping including patients with an LOS within a second range may be assigned a second accuracy rating; a third grouping including patients with an LOS within a third range may be assigned a third accuracy rating; and so on. The accuracy rating of each grouping may represent an accuracy of the CNN in predicting an LOS within the range associated with each grouping. For example, the CNN may have a high accuracy in predicting patient LOS for patients that are typically discharged around 2-3 days after they are admitted, but the CNN may have a lower accuracy in predicting patient LOS for patients that are typically discharged 10 days after they are admitted. The CNN may have an even lower accuracy in predicting patient LOS for patients that are typically discharged after several months. The accuracy of the CNN for different LOS ranges may be advantageously used to increase an accuracy of a predicted discharge date window of the patient, as described in greater detail below in reference to FIG. 8.

At 718, method 700 includes determining whether an overall performance of the CNN (e.g., across a plurality of groupings) is within a threshold accuracy. If at 718 it is determined that the CNN-based model is within the threshold accuracy, method 700 proceeds to 722. At 722, method 700 includes releasing the CNN-based model for deployment, and method 700 ends. If at 718 it is determined that the CNN-based model is not within the threshold accuracy, method 700 proceeds to 720.

At 720, method 700 may include reordering the groupings based on the accuracy rating ratings assigned at 716. For example, in a first training stage, training of the CNN on each grouping may be performed in a first order. For example, the first order may be based on an LOS range of each grouping, where a first grouping used to train the CNN corresponds to a first LOS range; a second grouping used to train the CNN corresponds to a second LOS range, where the second LOS range is longer than the first LOS range; and a third grouping used to train the CNN corresponds to a third LOS range, where the third LOS range is longer than the second LOS range. After the first training step has completed, to increase a performance of the CNN, the groupings may be reordered according to a new order based on a relative accuracy of the first, second, and third groupings. For example, the CNN may predict an LOS of patients in the second LOS range with the highest accuracy; the CNN may predict an LOS of patients in the first LOS range with a next highest accuracy; and the CNN may predict an LOS of patients in the third LOS range with a next highest accuracy. Based on the relative accuracies of the CNN, the groupings may be reordered such that the grouping associated with the second LOS range is first in the new order; the grouping associated with the first LOS range is second in the new order; and the grouping associated with the third LOS range is third in the new order. After reordering the groupings based on the accuracy ratings, method 700 may proceed back to 712, where the CNN may be retrained on each grouping in accordance with the new order.

In some embodiments, the CNN may be used to cluster similar patients together into new groupings, which may be used for further training.

By retraining the CNN in accordance with the new order of groupings, an overall accuracy of the CNN may be increased, where LOS's of new patient data vectors are predicted by the CNN with greater accuracy. Additionally, after retraining the CNN on each grouping in accordance with the new order, the relative accuracy of the CNN on the different LOS ranges may change. If the relative accuracy of the CNN on the different LOS ranges changes, the groupings may be once again reordered, and the CNN retrained on the groupings in the new order. In this way, the CNN may be trained iteratively and/or repeatedly to prioritize increasing an accuracy of the CNN on the groupings for which the CNN is most accurate.

In other embodiments, a different order of groupings may be used. For example, at 720, method 700 may include reordering the groupings in an increasing order of accuracy, where a first grouping of the order is a grouping on which the CNN is least accurate, followed by groupings on which the CNN is progressively more accurate. The CNN may then be retrained based on the new order, where the CNN may be trained iteratively and/or repeatedly to prioritize increasing and accuracy of the CNN on the groupings for which the CNN is least accurate.

Referring now to FIG. 8, a method 800 is shown for predicting patient discharge date windows for a selected plurality of patients, according to an embodiment of the disclosure. Method 800 may be carried out by a patient discharge date prediction system, such as patient discharge date prediction system 220 of FIG. 2, according to instructions stored in a memory (e.g., non-transitory memory 206) of the patient discharge date prediction system, which may be executed by a processor (e.g., processor(s) 204) of the patient discharge date prediction system. The patient discharge window predictions may be made using a patient LOS prediction model, such as patient LOS prediction model 102 of FIG. 1, which may predict a length of stay of a patient based on patient data. The patient LOS prediction model may be a CNN-based model trained as described in reference to LOS prediction model training system 600 of FIG. 6A, and deployed as described in relation to deployment diagram 650 of FIG. 6B.

Method 800 begins at operation 802, where method 800 includes receiving a selection of patients for whom to predict the discharge date windows. In some embodiments, the patient discharge date prediction system may be hosted on a server (e.g., of the healthcare organization) and accessible to a plurality of hospitals over a network via a patient management application. A user (e.g., a care provider) may enter in a name of a hospital or select a hospital from a list of hospitals into a UI of the patient management application (e.g., UI 300) in order to indicate a selection of patients associated with the hospital. In other embodiments, a relevant hospital may automatically be selected based on a pre-defined configuration file created during a setup stage of the patient discharge date prediction system. The user may also enter in a name of a hospital unit to view a selection of patients within the hospital unit.

The selection of patients may be a total number of patients of the hospital or hospital unit, or a subset of the patients of the hospital or hospital unit. In other words, to reduce a processing time and receive results more quickly, the user may select a small group of patients of the hospital to quickly determine which patients of the small group of patients may be ready to be discharged, rather than wait for results from a larger group of patients. For example, the user may reduce a number of patients to request discharge window predictions for by using one or more of the filters described in reference to UI 300 of FIG. 3. In other examples, a discharge window prediction may be generated for each patient in the hospital unit, and a selected subset of the discharge window predictions may be displayed via the user interface, based on the filtering/user selections described above.

At 804, method 800 includes receiving a desired level of confidence of the discharge window predictions from the user. The desired level of confidence may reflect an estimated level of confidence of the patient discharge date prediction system that the patient may be discharged within the predicted discharge date window. For example, if the desired confidence level is high (e.g., 80%), the length of the discharge window may be extended to include a wider range of dates, to achieve the desired level of confidence that the patient may be discharged within the discharge window. Alternatively, if the desired confidence level is lower (e.g., 50%), the length of the discharge window may be reduced to include a narrower range of dates. If the desired confidence level is very low (e.g., 10%), the predicted discharge date window may be the predicted discharge date.

In some embodiments, the desired level of confidence may be selected by the user, for example, via a menu displayed in the UI of the patient management application. The desired confidence level may also be pre-configured in the patient discharge date prediction system by an administrator of the hospital or the patient discharge date prediction system. For example, the pre-configured confidence level may be based on administrative guidelines of the hospital or hospital network.

At 806, method 800 includes extracting patient data corresponding to the selected patients from various data sources and generating a data vector representing the patient data. The data vector may include one or more encodings of the patient data, as described above in reference to FIG. 5B.

In various embodiments, the patient data may be retrieved from one or more EHR and/or other computer systems of the hospital and/or the healthcare organization, such as computer systems and databases 230 of FIG. 2. The other computer systems may include a local monitoring application of the hospital used to make routine measurements (e.g., current vital signs) or tests of the patient.

In some embodiments, a portion of a comprehensive set of patient data of the patient may be extracted, and other portions of patient data may not be extracted. For example, a first set of patient data may be a comprehensive set of data of the patient, and the first set of patient data may be stored across a plurality of computer systems (e.g., EHR systems) and/or databases of a healthcare system. Out of the first set of patient data, a second, smaller set of patient data (e.g., a subset) may be retrieved, where the second, smaller set of patient data is patient data determined by the patient discharge date prediction system to be most relevant to a patient discharge.

In some embodiments, vital signs or clinical markers may be retrieved from a single source, such as a single EHR system, where the patient discharge window prediction module may retrieve a plurality of records of the vital sign or clinical marker and select a most recent vital sign or clinical marker from the plurality of records. Further, the patient discharge window prediction module may analyze the plurality of records to determine one or more trends in vital signs or clinical markers. In some examples, the patient discharge date prediction system may receive a secure data feed from the EHR that includes the patient parameters (e.g., vital signs, ventilation status, and care provider-entered information such as Glasgow coma score, etc.) for the patient. By obtaining the patient parameters from a single source on a semi-continuous feed (e.g., updated every 10, 15, or 30 seconds), the efficiency of the patient discharge date prediction system may be increased by reducing processing demands and network traffic associated with obtaining the patient parameters from multiple sources and/or requesting the patient parameters each time a discharge window prediction is made. In some examples, the patient discharge date prediction system may map specific feeds for each data source from the EHR using an API.

At 808, method 800 includes generating a predicted discharge date for each patient of the selection of patients. At 810, generating the predicted discharge date for each patient includes inputting each data vector of each patient of the selected patients into the CNN-based LOS prediction model. Specifically, each data vector may be inputted into a first convolutional layer of a CNN of the CNN-based LOS prediction model. At 812, generating the predicted discharge date for each patient includes propagating input values of the data vectors through layers of the network, from the input layer, through one or more hidden layers, until reaching an output layer of the CNN, as described above in reference to method 700 of FIG. 7. At 814, generating the predicted discharge date for each patient may include performing convolutions and/or pooling operations on the data vector to generate an output of the CNN, where the output of the CNN includes a predicted discharge date of the patient.

At 816, method 800 includes generating a discharge window prediction for each patient of the selection of patients from the predicted discharge date outputted by the CNN-based LOS prediction model. In various embodiments, the discharge window prediction for each patient may be based on the level of confidence received from the user (or pre-configured) at 804. The discharge window prediction may be a date range during which the discharge date prediction system predicts the patient will be discharged, within a threshold probability, where the threshold probability is based on the level of confidence.

In some embodiments, the discharge window prediction may include a number of days surrounding the predicted discharge date, where the number of days is a function of the level of confidence. For example, if the desired level of confidence of the discharge date prediction system in the predicted discharge date window is low, the discharge window prediction may be a date range including a first small number of days prior to the predicted discharge date, and a second small number of days after the predicted discharge date. In various embodiments, the first small number may be the same as the second small number. Alternatively, if the desired level of confidence of the discharge date prediction system in the predicted discharge date window is high, the discharge window prediction may be a date range including a first, larger number of days prior to the predicted discharge date, and a second, larger number of days after the predicted discharge date, where the first, larger number may be the same as the second, larger number.

In other embodiments, the discharge window prediction may include a number of days surrounding the predicted discharge date, where the number of days is based partly on the level of confidence and partly on other information considered or generated by the discharge date prediction system. For example, in some embodiments, the CNN of the CNN-based LOS prediction model may include an additional output, where the additional output may indicate an estimated probability of the predicted LOS being accurate. In other embodiments, the predicted discharge date window may be partially based on a classification of the LOS of each patient. For example, an accuracy of the CNN-based LOS prediction model in predicting a discharge date of a patient may depend on a length of the LOS, as described above in reference to method 700 of FIG. 7. If a predicted LOS of a patient is associated with a classification with a high degree of accuracy, the predicted discharge date window may be adjusted to a more narrow range of dates. Alternatively, if the predicted LOS of a patient is associated with a classification with a low degree of accuracy, the predicted discharge date window may be adjusted to a wider range of dates.

At 818, method 800 includes displaying predicted discharge date window elements indicating the predicted discharge date windows of the selected patients on the display screen of the patient discharge date prediction system (e.g., of the patient management application). The predicted discharge date window element may be presented on the screen in a UI of the patient discharge date prediction system, for example, as described in reference to FIG. 3 above. The patients may be displayed in a list on the screen in a predetermined order, based on user-selected criteria and/or guidelines of the hospital. In some embodiments, the predicted discharge date window element of a patient may be displayed in a row (or column) corresponding to the patient and identified by a patient identifier (e.g., a name, ID, etc.). In other embodiments, displaying the predicted discharge date window elements of the selected patients on the display screen may include ranking the patients displayed on the display screen by discharge window prediction (e.g., displayed in a different order for ease of use). For example, one discharge window prediction may be earlier than another discharge window prediction, where the patient discharge date prediction system may display patients that are predicted to be discharged first (e.g., at a top of a list of discharge window predictions displayed on the display screen), followed by patients who may be discharged later, and with patients predicted to be discharged after a longer time frame towards or at the bottom of the list. In this way, a care provider may view at a glance a number of patients that may be discharged soon, or within a desired time frame, to free up resources of the hospital.

In some embodiments, the user may enter a user input to a predicted discharge date window element, such as a mouse or touch hover, or a mouse click or touch input, in order to view additional information associated with the discharge window prediction. If the user has selected a discharge window prediction or element, additional information may be displayed based on the input. Displaying the additional information may include displaying a pop-up display based on the user input. When a user selects a predicted discharge date window element for a patient, information relating to a discharge of the patient may be displayed. For example, in one embodiment, the predicted discharge date window element may be a compressed view that displays a predicted discharge date, and the discharge window may be displayed when the user hovers over the predicted discharge date window element. In another embodiment, an earliest discharge date of a predicted discharge date window may be displayed when the user hovers over the predicted discharge date window element.

Further, the information relating to the discharge patient may be displayed when the user selects the predicted discharge date window element while the patient discharge date prediction system is in an unlaunched state. For example, the patient management application may be accessible to the user when the patient discharge date prediction system is not operating. The user may request to view patient discharge date window predictions of a selection of patients, whereby for each selected patient, a most recently predicted discharge date window may be displayed in the patient management application. For example, the most recently predicted discharge date window may have been generated during a previous operation of the patient discharge date prediction system (e.g., on a previous day). Patient information available at the time when the most recently predicted discharge date window was generated may be stored in the patient management application, such that the patient information may be displayed in the UI in response to the user selecting a discharge date prediction element of the UI. For example, when the user hovers over the predicted discharge date window element, an earliest discharge date of a predicted discharge date window may be displayed. If the user selects the earliest discharge date, additional patient data related to the predicted discharge date window may be displayed in the UI (e.g., via a pop-up window).

In addition to displaying the predicted discharge date window elements representing the discharge window predictions on the display screen, the discharge window predictions may be stored in a database of the hospital or hospital network (e.g., computer systems and databases 230 of FIG. 2).

Referring now to FIGS. 9A through 14B, a second embodiment of the patient discharge date prediction system is shown, where the patient discharge date prediction system generates probabilistic discharge timing predictions in addition to the predicted discharge date described above in reference to FIGS. 1 through 8. In the second embodiment, the patient discharge date prediction system generates a discharge horizon probability distribution over possible discharge times, for example, indicating relative probabilities that a patient will be discharged on a specified date, the day after the specified date, or two or more days from the specified date, and a time-of-day discharge probability distribution indicating relative probabilities that the patient will be discharged within a first time window on the discharge day, a second time window on the discharge day, or a third time window on the discharge day. The second embodiment of the patient discharge date prediction system further applies Wilson score interval calibration to the discharge horizon probability distribution and the time-of-day discharge probability distribution to generate calibrated probabilities and associated confidence intervals for each probability. The calibrated probabilities and confidence intervals may be displayed in a user interface of the patient discharge date prediction system, as described in greater detail below.

FIG. 9A shows a data flow diagram of a patient discharge date prediction system 900 in accordance with the second embodiment. Patient discharge date prediction system 900 may be a non-limiting example of patient discharge date prediction system 100 of FIG. 1, and elements of patient discharge date prediction system 900 having similar names to elements of patient discharge date prediction system 100 may be non-limiting examples of the similarly named elements of patient discharge date prediction system 100. Elements of patient discharge date prediction system 900 that are included in patient discharge date prediction system 100 of FIG. 1 and that have been described above in reference to FIG. 1 are not described again here.

Patient discharge date prediction system 900 may receive patient data of a patient 901 from a plurality of data sources. The patient data may include admission data 910, ICD and Elixhauser codes 912, insurance and demographics data 914, patient flow data 916, surgery and special condition data 918, and labs, vitals, and imaging data 920, as described above in reference to FIG. 1. In addition to the clinical patient data categories described in reference to FIG. 1, patient discharge date prediction system 900 may receive operational workflow data 922 from one or more operational systems of the healthcare facility. Operational workflow data 922 may include data from systems that influence a time of day at which patient discharges occur. For example, operational workflow data 922 may include rounding schedule data indicating times at which attending physicians conduct patient rounds on a given unit, as discharge orders are frequently entered during or shortly after physician rounding. Operational workflow data 922 may include order entry timing data from one or more computerized physician order entry (CPOE) systems of the healthcare facility, indicating historical patterns of when discharge orders, medication reconciliation orders, and follow-up appointment orders are entered for patients of the healthcare facility. Operational workflow data 922 may include consult completion timing data indicating average durations and completion times of pending consults that may represent barriers to discharge. Operational workflow data 922 may include placement logistics data indicating lead times and scheduling constraints associated with post-acute placement services such as skilled nursing facilities, rehabilitation facilities, and home health agencies. Operational workflow data 922 may further include transport scheduling data indicating availability windows and scheduling patterns of patient transport services. In this way, operational workflow data 922 may capture workflow-driven patterns that affect actual discharge timing, such as a tendency for discharges to cluster in the afternoon following morning rounds, or a tendency for discharges requiring post-acute placement to occur later in the day due to coordination delays.

The patient data of patient 901, including operational workflow data 922, may be received at a patient LOS prediction model 902. Patient LOS prediction model 902 may be a trained neural network model, such as a CNN, that receives the patient data as input and generates a plurality of outputs. In the second embodiment, patient LOS prediction model 902 may generate three outputs simultaneously during a single forward pass of the patient data through the neural network.

A first output of patient LOS prediction model 902 may be a predicted length of stay 904. Predicted length of stay 904 may be a number of days that patient 901 is expected to remain a patient of the healthcare facility, as described above in reference to predicted LOS 104 of FIG. 1. Predicted length of stay 904 may be provided to a date calculator 906, which may calculate a predicted discharge date of patient 901 based on predicted length of stay 904 and a current date.

A second output of patient LOS prediction model 902 may be a discharge horizon probability distribution 930 over possible discharge times. Discharge horizon probability distribution 930 may indicate a probability that patient 901 will be discharged on a specified date, such as a current date, a probability that patient 901 will be discharged the day after the specified date, and a probability that patient 901 will be discharged two or more days from the specified date. In various embodiments, the probabilities of discharge horizon probability distribution 930 may sum to one, as a result of being generated by a softmax output layer of patient LOS prediction model 902. For example, discharge horizon probability distribution 930 may indicate a 72% probability that patient 901 will be discharged today, a 20% probability that patient 901 will be discharged tomorrow, and an 8% probability that patient 901 will be discharged two or more days from the current date.

A third output of patient LOS prediction model 902 may be a time-of-day probability distribution 932. Time-of-day probability distribution 932 may indicate a probability that patient 901 will be discharged within a first time window (e.g., 12 AM to 11 AM), a probability that patient 901 will be discharged within a second time window (e.g., 12 PM to 6 PM), and a probability that patient 901 will be discharged within a third time window (e.g., 7 PM to 11 PM). In various embodiments, the probabilities of time-of-day probability distribution 932 may sum to one, as a result of being generated by a separate softmax output layer of patient LOS prediction model 902. Time-of-day probability distribution 932 may reflect not only a clinical readiness of patient 901 for discharge but also operational and logistical factors captured in operational workflow data 922 that influence when the discharge is likely to be executed within a given day.

Discharge horizon probability distribution 930 and time-of-day probability distribution 932 may be provided to a Wilson score interval calibration 934. Wilson score interval calibration 934 may apply a statistical calibration methodology to the probabilities of discharge horizon probability distribution 930 and time-of-day probability distribution 932 to generate calibrated probabilities with associated confidence intervals. Wilson score interval calibration 934 may receive historical observed discharge rates 936 as an additional input. Historical observed discharge rates 936 may represent actual discharge rates observed for patients binned into predicted probability bands during a calibration phase performed on historical patient data of the healthcare facility. For example, during the calibration phase, patients may be grouped into predicted probability bands (e.g., 0-10%, 10-20%, 20-30%, and so on), and an actual discharge rate may be computed for patients within each band. The Wilson score interval may then be calculated for each band based on the computed actual discharge rate and a number of patients within the band.

During inference, Wilson score interval calibration 934 may receive a predicted probability from discharge horizon probability distribution 930 or time-of-day probability distribution 932, identify a predicted probability band corresponding to the predicted probability, and retrieve a stored Wilson score interval associated with the identified predicted probability band. The Wilson score interval may include a lower confidence bound and an upper confidence bound around an observed discharge rate for the identified predicted probability band. In this way, Wilson score interval calibration 934 may provide a statistically validated range indicating how reliable the predicted probability has been historically for patients in a similar predicted probability band.

Wilson score interval calibration 934 may output calibrated horizon probabilities and confidence intervals 940 and calibrated time-of-day probabilities and confidence intervals 942. Calibrated horizon probabilities and confidence intervals 940 may include, for each category of discharge horizon probability distribution 930 (e.g., on a current date, on the day after the current date, two or more days after the current date), a calibrated probability value and a lower confidence bound and an upper confidence bound derived from the Wilson score interval. Calibrated time-of-day probabilities and confidence intervals 942 may include, for each category of time-of-day probability distribution 932 (e.g., morning, afternoon, evening), a calibrated probability value and a lower confidence bound and an upper confidence bound derived from the Wilson score interval. Calibrated horizon probabilities and confidence intervals 940 and calibrated time-of-day probabilities and confidence intervals 942 may be provided to a user interface of the patient discharge date prediction system for display to a care provider, as described in greater detail below in reference to FIGS. 9B, 10, and 11.

FIG. 9B shows a block diagram 950 illustrating a flow of data from the outputs of patient discharge date prediction system 900 of FIG. 9A into a patient manager GUI display 952 for rendering as user interface elements. Patient manager GUI display 952 may be a non-limiting example of a display generated by patient discharge date prediction system 220 of FIG. 2 for display on display device 201 of FIG. 2. Patient manager GUI display 952 may receive three inputs from the prediction and calibration pipeline described above in reference to FIG. 9A, and may render the three inputs as four distinct user interface elements for display to a care provider.

A first input to patient manager GUI display 952 may be predicted discharge date 950, as generated by date calculator 906 of FIG. 9A based on predicted length of stay 904. Predicted discharge date 950 may be the same as or similar to predicted discharge date 150 of FIG. 1, and may indicate a specific calendar date on which patient 901 is predicted to be discharged from the healthcare facility. In various embodiments, predicted discharge date 950 may be expressed as an absolute date (e.g., “May 6, 2022”) or as a relative date (e.g., “In 3 days”).

A second input to patient manager GUI display 952 may be calibrated horizon probabilities and confidence intervals 940, as generated by Wilson score interval calibration 934 of FIG. 9A. Calibrated horizon probabilities and confidence intervals 940 may include, for each category of discharge horizon probability distribution 930 (e.g., on a current date, on the day after the current date, two or more days after the current date), a calibrated probability value and a lower confidence bound and an upper confidence bound derived from the Wilson score interval, as described above in reference to FIG. 9A.

A third input to patient manager GUI display 952 may be calibrated time-of-day probabilities and confidence intervals 942, as generated by Wilson score interval calibration 934 of FIG. 9A. Calibrated time-of-day probabilities and confidence intervals 942 may include, for each category of time-of-day probability distribution 932 (e.g., morning, afternoon, evening), a calibrated probability value and a lower confidence bound and an upper confidence bound derived from the Wilson score interval, as described above in reference to FIG. 9A.

Patient manager GUI display 952 may receive the three inputs and render the three inputs as four user interface elements. A first user interface element may be a predicted discharge date window element 954. Predicted discharge date window element 954 may display predicted discharge date 950 in the user interface, for example, as a date range or a relative time indicator (e.g., “In 2-3 Days”). Predicted discharge date window element 954 may be similar to predicted discharge date window element 362 described above in reference to FIG. 3, and may be displayed in a dates column of the user interface adjacent to other date-related information of the patient such as an admission date, a geometric mean length of stay, and an expected discharge date.

A second user interface element may be a discharge horizon probability element 956. Discharge horizon probability element 956 may display the calibrated discharge horizon probabilities from calibrated horizon probabilities and confidence intervals 940 in the user interface. For example, discharge horizon probability element 956 may display a probability that the patient will be discharged on a current date, a probability that the patient will be discharged the day after the current date, and a probability that the patient will be discharged two or more days from the current date. In some embodiments, discharge horizon probability element 956 may be displayed as a tooltip or pop-up element that appears in response to a user interaction with a control of the user interface, such as a hover or click on a discharge forecast button associated with the patient. For example, discharge horizon probability element 956 may display text such as “Today: 72%; Tomorrow: 20%; 2+ Days: 8%” in a tooltip adjacent to the discharge forecast button.

A third user interface element may be a time-of-day probability element 958. Time-of-day probability element 958 may display the calibrated time-of-day discharge probabilities from calibrated time-of-day probabilities and confidence intervals 942 in the user interface. For example, time-of-day probability element 958 may display a probability that the patient will be discharged within a first time window, a probability that the patient will be discharged within a second time window, and a probability that the patient will be discharged within a third time window. In some embodiments, time-of-day probability element 958 may be displayed as a tooltip or pop-up element that appears in response to a user interaction with a control of the user interface, such as a hover or click on an estimated discharge date button associated with the patient. For example, time-of-day probability element 958 may display text such as “AM: 72%; PM: 20%; Evening: 8%” in a tooltip adjacent to the estimated discharge date button.

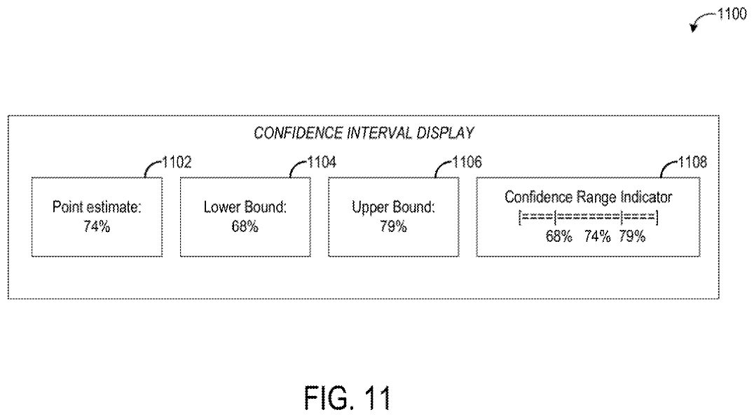

A fourth user interface element may be a confidence interval display 960. Confidence interval display 960 may display the confidence intervals associated with the calibrated probabilities from calibrated horizon probabilities and confidence intervals 940 and calibrated time-of-day probabilities and confidence intervals 942. In various embodiments, confidence interval display 960 may include a point estimate, a lower confidence bound, and an upper confidence bound for a selected probability category, as well as a graphical confidence range indicator that visually depicts the range between the lower confidence bound and the upper confidence bound. Confidence interval display 960 may be displayed in response to a user interaction with discharge horizon probability element 956 or time-of-day probability element 958, such as a selection of a specific probability category within one of the tooltip elements. For example, if a care provider selects the “Today: 72%” probability displayed in discharge horizon probability element 956, confidence interval display 960 may appear showing a point estimate of 74%, a lower bound of 68%, and an upper bound of 79%, along with a graphical bar indicating the confidence range. Confidence interval display 960 is described in greater detail below in reference to FIG. 11.

By generating discharge horizon probability distribution 930 and time-of-day probability distribution 932 in addition to predicted length of stay 904, and by applying Wilson score interval calibration 934 to generate calibrated probabilities with confidence intervals, patient discharge date prediction system 900 may provide care providers with a more informative and transparent view of discharge timing than a single predicted discharge date alone. The care provider may use the calibrated probabilities and confidence intervals to prioritize discharge-related activities across a patient census based on the relative likelihood of discharge occurring within specific time windows, and may assess the reliability of the predictions based on the width of the confidence intervals. Further, by incorporating operational workflow data 922 into the prediction, patient discharge date prediction system 900 may account for operational and logistical factors that influence actual discharge timing, thereby increasing an accuracy of the time-of-day probability distribution 932 relative to a prediction based solely on clinical data.

By rendering the outputs of the prediction and calibration pipeline as four distinct user interface elements within patient manager GUI display 952, the patient discharge date prediction system may provide care providers with multiple levels of discharge timing information in a layered manner. A care provider may view predicted discharge date window element 954 at a glance to determine an approximate discharge date of the patient, and may interact with discharge horizon probability element 956 and time-of-day probability element 958 to obtain more granular probabilistic information about when the discharge is likely to occur. Further, the care provider may access confidence interval display 960 to assess a reliability of the probabilistic predictions based on the width of the confidence intervals. In this way, patient manager GUI display 952 may present discharge timing information at progressively increasing levels of detail, allowing care providers to access the level of information appropriate to their current decision-making demands without overwhelming the user interface with excessive data. In various embodiments, the discharge horizon probability distribution and the time-of-day discharge probability distribution may be generated and displayed independently of one another. For example, in some embodiments, patient discharge date prediction system 900 may generate and display only the discharge horizon probability distribution without generating or displaying the time-of-day discharge probability distribution. In such embodiments, patient manager GUI display 952 may render discharge horizon probability element 956 without rendering time-of-day probability element 958, and the care provider may view the calibrated discharge horizon probabilities indicating relative probabilities of discharge on a current date, on the day after the current date, two or more days after the current date, without viewing time-of-day discharge probability information. Similarly, in other embodiments, patient discharge date prediction system 900 may generate and display only the time-of-day discharge probability distribution without generating or displaying the discharge horizon probability distribution. In such embodiments, patient manager GUI display 952 may render time-of-day probability element 958 without rendering discharge horizon probability element 956, and the care provider may view the calibrated time-of-day discharge probabilities indicating relative probabilities of discharge within the first, second, or third time windows, without viewing discharge horizon probability information. In still other embodiments, both the discharge horizon probability distribution and the time-of-day discharge probability distribution may be generated and displayed together, as described above. The selection of which probability distributions to generate and display may be configurable by an administrator of the healthcare facility or by a care provider via a settings menu of UI 300 (e.g., via settings button 309). For example, a first healthcare facility may configure patient discharge date prediction system 900 to display only the discharge horizon probability distribution, a second healthcare facility may configure patient discharge date prediction system 900 to display only the time-of-day discharge probability distribution, and a third healthcare facility may configure patient discharge date prediction system 900 to display both distributions. In embodiments where only one of the two distributions is generated, patient LOS prediction model 902 may include only the corresponding output branch (e.g., only the softmax output layer for the discharge horizon probability distribution, or only the softmax output layer for the time-of-day probability distribution), or patient LOS prediction model 902 may include both output branches but patient discharge date prediction system 900 may suppress display of the distribution that is not configured for display. In this way, the discharge horizon probability distribution and the time-of-day discharge probability distribution are independent prediction outputs that may be individually included in or excluded from the user interface of the patient discharge date prediction system based on the operational uses of the healthcare facility.

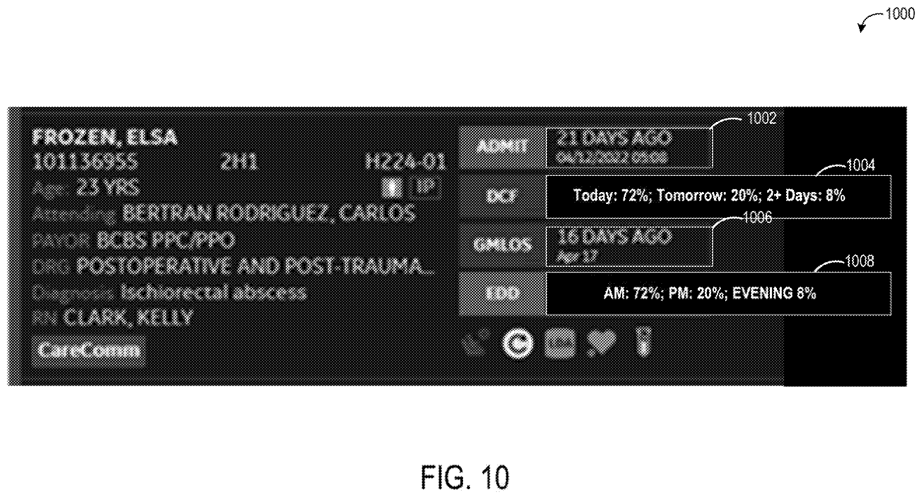

Referring now to FIG. 10, a screenshot of a dates panel 1000 is shown in accordance with the second embodiment. Dates panel 1000 may be a non-limiting example of dates panel 336 of UI 300 described above in reference to FIG. 3, and may be displayed in dates column 318 of UI 300 for a patient of the patient census list view. Dates panel 1000 may include similar elements as described in reference to dates panel 336, such as admit graphical element 360 and a GMLOS element. In the second embodiment shown in FIG. 10, dates panel 1000 may include discharge prediction elements that display the calibrated discharge horizon probabilities and calibrated time-of-day discharge probabilities generated by patient discharge date prediction system 900 of FIG. 9A, in place of or in addition to the discharge prediction elements described above in reference to dates panel 336 of FIG. 3.

In the embodiment shown in FIG. 10, dates panel 1000 includes an admit display element 1002, a discharge horizon probability element 1004 (e.g., discharge horizon probability element 1004), a GMLOS display element 1006, and a time-of-day probability display element 1008 (e.g., time-of-day probability display element 958). In this embodiment, discharge horizon probability element 1004 and time-of-day probability display element 1008 may replace predicted discharge date window element 362 and expected discharge date element 364 described above in reference to dates panel 336 of FIG. 3, respectively. In other embodiments, discharge horizon probability element 1004 and time-of-day probability display element 1008 may be displayed alongside predicted discharge date window element 362 and expected discharge date element 364, rather than replacing them. In still other embodiments, discharge horizon probability element 1004 and time-of-day probability display element 1008 may replace or be displayed alongside any of the other date-related elements described in reference to dates panel 336 of FIG. 3, such as predicted discharge date 366. In further embodiments, the discharge horizon probability information and the time-of-day probability information may be displayed as tooltip pop-up elements that appear in response to a user interaction with an existing element of dates panel 336, such as a hover or click on predicted discharge date window element 362 or expected discharge date element 364, rather than being displayed as separate buttons in dates panel 1000.

Discharge horizon probability element 1004 may display the calibrated discharge horizon probabilities from calibrated horizon probabilities and confidence intervals 940 of FIG. 9A. In the depicted example, discharge horizon probability element 1004 displays text indicating “Today: 72%; Tomorrow: 20%; 2+ Days: 8%”, where 72% represents a calibrated probability that the patient will be discharged today, 20% represents a calibrated probability that the patient will be discharged tomorrow, and 8% represents a calibrated probability that the patient will be discharged two or more days from the current date.

Time-of-day probability display element 1008 may display the calibrated time-of-day discharge probabilities from calibrated time-of-day probabilities and confidence intervals 942 of FIG. 9A. For example, time-of-day probability display element 1008 may display text indicating “AM: 72%; PM: 20%; EVENING 8%”, where 72% represents a calibrated probability that the patient will be discharged within a morning time window (e.g., 12 AM to 11 AM), 20% represents a calibrated probability that the patient will be discharged within an afternoon time window (e.g., 12 PM to 6 PM), and 8% represents a calibrated probability that the patient will be discharged within an evening time window (e.g., 7 PM to 11 PM).

In various embodiments, the probabilities displayed in discharge horizon probability element 1004 and time-of-day probability display element 1008 may be the calibrated probabilities generated by Wilson score interval calibration 934 of FIG. 9A, rather than the raw probabilities output by patient LOS prediction model 902. In other embodiments, the raw probabilities output by patient LOS prediction model 902 may be displayed in discharge horizon probability element 1004 and time-of-day probability display element 1008, and the calibrated probabilities and confidence intervals may be accessible via a further user interaction, such as a selection of a specific probability category within the tooltip, as described in greater detail below in reference to FIG. 11.

In some embodiments, the probabilities displayed in discharge horizon probability element 1004 and time-of-day probability display element 1008 may be updated periodically or in response to new patient data becoming available, as described above in reference to FIG. 9A. For example, if new lab results or vital sign data are recorded for the patient, patient LOS prediction model 902 may generate updated discharge horizon probabilities and time-of-day probabilities, Wilson score interval calibration 934 may generate updated calibrated probabilities and confidence intervals, and the updated calibrated probabilities may be reflected in discharge horizon probability element 1004 and time-of-day probability display element 1008.

By displaying the calibrated discharge horizon probabilities and time-of-day discharge probabilities within dates panel 1000, the patient discharge date prediction system may allow care providers to quickly assess discharge timing likelihoods for individual patients without navigating away from the patient census list view of UI 300. A care provider may scan the patient census list view and view or interact with discharge horizon probability element 1004 and time-of-day probability display element 1008 for any patient of interest to view the probabilistic discharge timing information for that patient. In this way, the probabilistic discharge timing information may be accessible on demand without consuming additional screen space in dates panel 1000 when not actively being viewed.

FIG. 11 shows a confidence interval display 1100 in accordance with the second embodiment of FIG. 10. Confidence interval display 1100 may be a non-limiting example of confidence interval display 960 of FIG. 9B. In the embodiment shown in FIG. 11, confidence interval display 1100 may be generated as a pop-up element that appears in response to a user interaction with a specific probability category displayed in discharge horizon probability element 1004 and time-of-day probability display element 1008 of FIG. 10. For example, if a care provider selects the “Today: 72%” probability displayed in discharge horizon probability element 1004, confidence interval display 1100 may appear as a pop-up element adjacent to or overlaying discharge horizon probability element 1004. In other embodiments, confidence interval display 1100 may be integrated into the main display of patient manager graphical user interface 300 in a different manner, such as being displayed inline within discharge horizon probability element 1004 or time-of-day probability display element 1008, being displayed in a dedicated panel or sidebar of patient manager GUI 300, or being displayed within dates panel 1000 adjacent to the corresponding probability value.

Confidence interval display 1100 may include a point estimate element 1102, a lower bound element 1104, an upper bound element 1106, and a confidence range indicator 1108. Point estimate element 1102 may display a calibrated point estimate of the selected probability category. The calibrated point estimate may represent an observed discharge rate for patients in a predicted probability band corresponding to the selected probability category, as computed during the calibration phase described above in reference to Wilson score interval calibration 934 of FIG. 9A. For example, point estimate element 1102 may display “Point estimate: 74%”, indicating that historically, 74% of patients in the predicted probability band corresponding to the selected probability category were actually discharged within the corresponding time window.

Lower bound element 1104 may display a lower confidence bound derived from the Wilson score interval for the selected probability category. For example, lower bound element 1104 may display “Lower Bound: 68%”, indicating that the lower bound of the Wilson score interval for the observed discharge rate is 68%. Upper bound element 1106 may display an upper confidence bound derived from the Wilson score interval for the selected probability category. For example, upper bound element 1106 may display “Upper Bound: 79%”, indicating that the upper bound of the Wilson score interval for the observed discharge rate is 79%.

Confidence range indicator 1108 may display a graphical representation of the confidence interval, including a visual bar or range indicator that depicts the span between the lower confidence bound and the upper confidence bound, with the point estimate indicated at a position within the span. For example, confidence range indicator 1108 may display a horizontal bar extending from 68% to 79%, with a marker at 74% indicating the point estimate. Confidence range indicator 1108 may allow the care provider to visually assess a width of the confidence interval, where a narrow confidence interval may indicate a high degree of reliability of the prediction and a wide confidence interval may indicate a lower degree of reliability.

The Wilson score interval displayed in confidence interval display 1100 may be calculated based on a number of historical patients in the predicted probability band and the observed discharge rate within the band. In various embodiments, the Wilson score interval may be a 95% confidence interval, although other confidence levels (e.g., 90%, 99%) may be used in different embodiments. The Wilson score interval may account for small sample sizes by providing asymmetric bounds around the observed rate, which may be advantageous when the number of historical patients in a given predicted probability band is relatively small.

In some embodiments, confidence interval display 1100 may include additional information, such as a number of historical patients used to compute the Wilson score interval, a date range over which the historical data was collected, or a last calibration date indicating when the Wilson score intervals were most recently updated. In other embodiments, confidence interval display 1100 may include a textual interpretation of the confidence interval, such as “Based on historical data, patients with similar predictions were discharged within this time window between 68% and 79% of the time.”

By providing confidence interval display 1100, the patient discharge date prediction system may allow care providers to assess a reliability of individual probability predictions on demand. A care provider may use the width of the confidence interval to determine how much weight to place on a given probability prediction when making discharge planning decisions. For example, a narrow confidence interval (e.g., 68% to 79%) may indicate that the prediction has been historically reliable for patients in a similar predicted probability band, and the care provider may rely on the prediction with a high degree of confidence. Alternatively, a wide confidence interval (e.g., 40% to 85%) may indicate that the prediction has been less reliable historically, and the care provider may seek additional clinical information before acting on the prediction. In this way, confidence interval display 1100 may provide a transparent and statistically grounded basis for clinical decision-making around discharge timing.

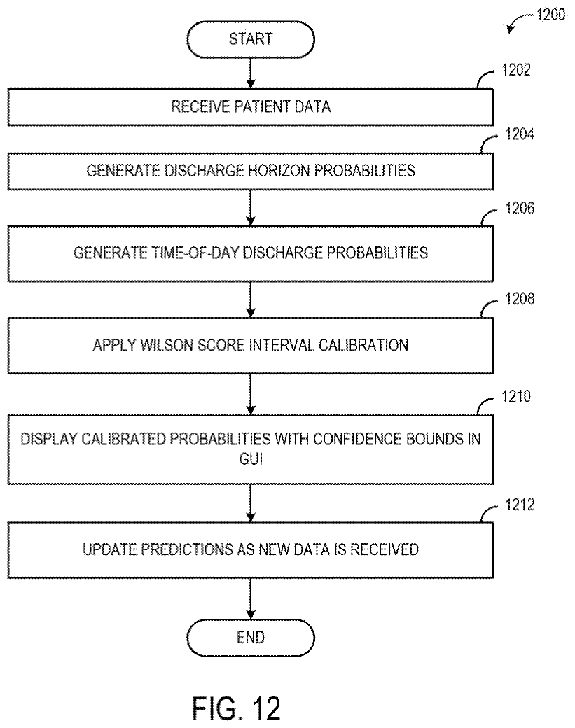

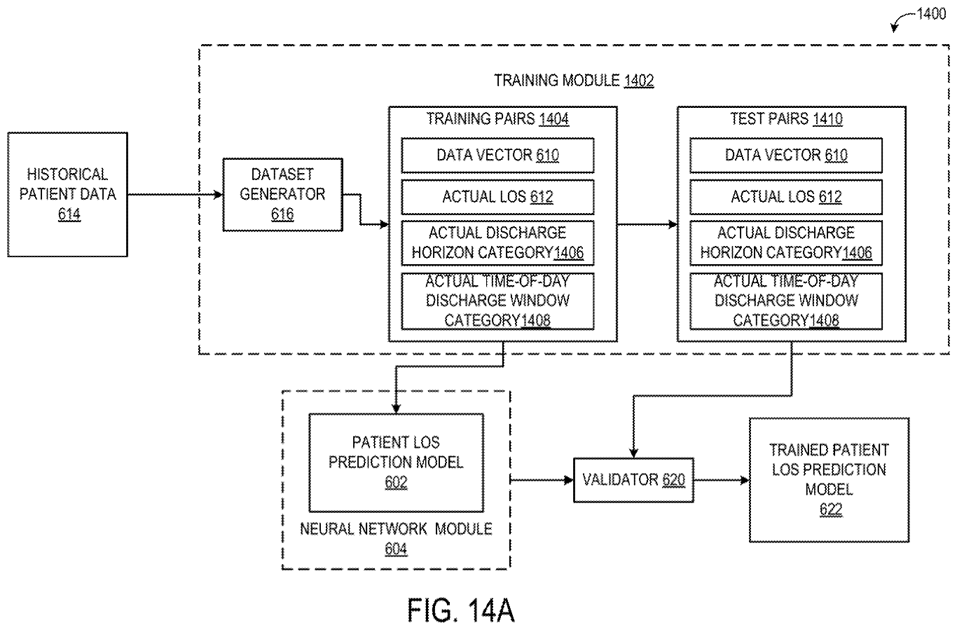

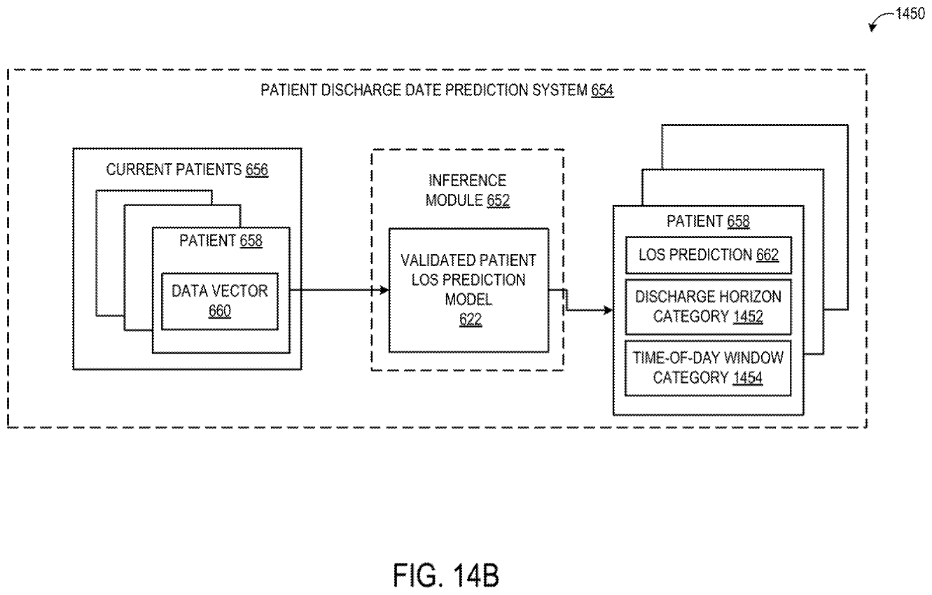

Referring now to FIG. 12, a flowchart of a method 1200 is shown for generating and displaying calibrated patient discharge probability predictions in accordance with the second embodiment. Method 1200 may be carried out by a patient discharge date prediction system, such as patient discharge date prediction system 900 of FIG. 9A, according to instructions stored in a non-transitory memory (e.g., non-transitory memory 206 of FIG. 2) of the patient discharge date prediction system, which may be executed by a processor (e.g., processor 204 of FIG. 2) of the patient discharge date prediction system. Method 1200 may be performed for each patient of a set of current patients (e.g., current patients 656 of FIG. 14B) for whom discharge predictions are to be generated. In various embodiments, method 1200 may be performed periodically, at scheduled times during a day, or in response to new patient data becoming available, as described above in reference to patient discharge date prediction system 220 of FIG. 2.

Method 1200 begins at 1202, where method 1200 includes receiving patient data of a patient of the healthcare facility. The patient data may be received from a plurality of data sources communicably coupled to the patient discharge date prediction system, as described above in reference to FIG. 9A. For example, the patient data may include admission data 910, ICD and Elixhauser codes 912, insurance and demographics data 914, patient flow data 916, surgery and special condition data 918, labs, vitals, and imaging data 920, and operational workflow data 922. The patient data may be received via one or more secure feeds from one or more electronic health record databases and other computer systems of the healthcare facility, as described above in reference to FIG. 1. In various embodiments, receiving the patient data at operation 1202 may include extracting the patient data from the plurality of data sources and generating a data vector (e.g., data vector 660 of FIG. 14B) representing the patient data, as described above in reference to data vector 552 of FIG. 5B. The data vector may encode the patient data from each of the plurality of data sources, including operational workflow data 922, into a format suitable for input into patient LOS prediction model 902 of FIG. 9A.

At 1204, method 1200 includes generating discharge horizon probabilities for the patient. Generating the discharge horizon probabilities may include inputting the data vector of the patient into patient LOS prediction model 902 of FIG. 9A and receiving, as an output of patient LOS prediction model 902, a discharge horizon probability distribution 930 indicating a probability that the patient will be discharged on a specified date (e.g., today), a probability that the patient will be discharged the day after the specified date, and a probability that the patient will be discharged two or more days from the specified date. In various embodiments, the discharge horizon probabilities may be generated by a softmax output layer of patient LOS prediction model 902, such that the probabilities of discharge horizon probability distribution 930 sum to one. For example, patient LOS prediction model 902 may output a discharge horizon probability distribution 930 indicating a 72% probability that the patient will be discharged today, a 20% probability that the patient will be discharged tomorrow, and an 8% probability that the patient will be discharged two or more days from the current date. In various embodiments, generating the discharge horizon probabilities at operation 1204 may be performed simultaneously with generating a predicted length of stay 904 of the patient, as described above in reference to FIG. 9A, where patient LOS prediction model 902 generates multiple outputs during a single forward pass of the data vector through the neural network. At 1206, method 1200 includes generating time-of-day discharge probabilities for the patient. Generating the time-of-day discharge probabilities may include receiving, as a further output of patient LOS prediction model 902 of FIG. 9A, a time-of-day probability distribution 932 indicating a probability that the patient will be discharged within a morning time window (e.g., 12 AM to 11 AM), a probability that the patient will be discharged within an afternoon time window (e.g., 12 PM to 6 PM), and a probability that the patient will be discharged within an evening time window (e.g., 7 PM to 11 PM). In various embodiments, the time-of-day discharge probabilities may be generated by a separate softmax output layer of patient LOS prediction model 902, such that the probabilities of time-of-day probability distribution 932 sum to one. The time-of-day discharge probabilities may reflect both a clinical readiness of the patient for discharge and operational and logistical factors captured in operational workflow data 922, such as rounding schedule data, order entry timing data, consult completion timing data, placement logistics data, and transport scheduling data, as described above in reference to FIG. 9A. In various embodiments, generating the time-of-day discharge probabilities at operation 1206 may be performed during the same forward pass of the data vector through patient LOS prediction model 902 as generating the discharge horizon probabilities at operation 1204. In various embodiments, operations 1204 and 1206 may be performed independently of one another. For example, in some embodiments, method 1200 may include generating the discharge horizon probabilities at operation 1204 without generating the time-of-day discharge probabilities at operation 1206, or generating the time-of-day discharge probabilities at operation 1206 without generating the discharge horizon probabilities at operation 1204, depending on a configuration of the patient discharge date prediction system. In such embodiments, the Wilson score interval calibration at operation 1208 may be applied only to the probability distribution that was generated, and the display at operation 1210 may include only the corresponding probability element (e.g., only discharge horizon probability element 956, or only time-of-day probability element 958).

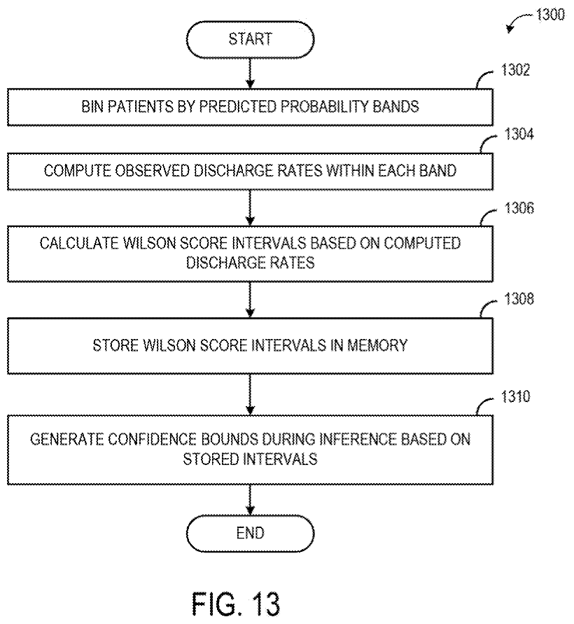

At 1208, method 1200 includes applying Wilson score interval calibration to the discharge horizon probabilities and the time-of-day discharge probabilities. In some examples, any statistically adjusted scoring or ranking metric that incorporates uncertainty can be applied. Applying Wilson score interval calibration may include performing the operations of Wilson score interval calibration 934 of FIG. 9A on discharge horizon probability distribution 930 and time-of-day probability distribution 932. In various embodiments, applying Wilson score interval calibration at operation 1208 may include, for each probability of discharge horizon probability distribution 930 and time-of-day probability distribution 932, identifying a predicted probability band corresponding to the probability, retrieving a stored Wilson score interval associated with the identified predicted probability band from historical observed discharge rates 936, and generating a calibrated probability and associated confidence interval based on the stored Wilson score interval. The Wilson score interval may include a lower confidence bound and an upper confidence bound around an observed discharge rate for the identified predicted probability band, as described above in reference to FIG. 9A. The process by which the Wilson score intervals are generated and stored is described in greater detail below in reference to method 1300 of FIG. 13. For example, if discharge horizon probability distribution 930 indicates a 72% probability that the patient will be discharged today, Wilson score interval calibration 934 may identify a predicted probability band of 70% to 80% corresponding to the 72% probability. Wilson score interval calibration 934 may then retrieve a stored Wilson score interval for the 70% to 80% predicted probability band, which may indicate that historically, patients in the 70% to 80% predicted probability band for same-day discharge were actually discharged on the same day at a rate of 74%, with a lower confidence bound of 68% and an upper confidence bound of 79%. The calibrated probability for the same-day discharge category may then be 74%, with a confidence interval of 68% to 79%. This process may be repeated for each probability category of discharge horizon probability distribution 930 and time-of-day probability distribution 932, to generate calibrated horizon probabilities and confidence intervals 940 and calibrated time-of-day probabilities and confidence intervals 942.

At 1210, method 1200 includes displaying the calibrated probabilities with confidence bounds in a graphical user interface of the patient discharge date prediction system. Displaying the calibrated probabilities with confidence bounds may include rendering the calibrated probabilities and confidence intervals as user interface elements within patient manager GUI display 952 of FIG. 9B, as described above in reference to FIGS. 9B, 10, and 11. For example, displaying the calibrated probabilities with confidence bounds at operation 1210 may include displaying a discharge horizon probability element 956 showing the calibrated discharge horizon probabilities (e.g., “Today: 72%; Tomorrow: 20%; 2+ Days: 8%”), displaying a time-of-day probability element 958 showing the calibrated time-of-day discharge probabilities (e.g., “AM: 72%; PM: 20%; EVENING 8%”), and displaying a confidence interval display 1100 of FIG. 11 showing a point estimate, a lower confidence bound, an upper confidence bound, and a confidence range indicator.

In various embodiments, displaying the calibrated probabilities with confidence bounds at operation 1210 may further include displaying a predicted discharge date window element 954 in dates panel 1000, where predicted discharge date window element 954 indicates a predicted discharge date of the patient based on predicted length of stay 904 generated by patient LOS prediction model 902 at operations 1204 and 1206. In this way, the care provider may view both the predicted discharge date and the probabilistic discharge timing information within a single dates panel 1000 of the patient census list view.

In various embodiments, displaying the calibrated probabilities with confidence bounds at operation 1210 may further include determining whether one or more probabilities of the discharge horizon probability distribution or the time-of-day discharge probability distribution exceed one or more predetermined threshold probabilities, and in response to determining that a probability exceeds a corresponding predetermined threshold probability, automatically initiating one or more discharge preparation workflows for the patient. For example, in response to determining that a probability of the discharge horizon probability distribution indicating that the patient will be discharged on a current date exceeds a predetermined threshold probability, the patient discharge date prediction system may automatically initiate one or more discharge preparation workflows including generating a notification to one or more care providers of the patient indicating one or more administrative tasks to be performed prior to discharging the patient, arranging transportation for the patient, initiating paperwork associated with discharging the patient, and generating a message to coordinate staffing, pharmacy, or post-acute placement services based on the time-of-day discharge probability distribution. Further, in response to determining that a probability of the time-of-day discharge probability distribution indicating that the patient will be discharged within a particular time window (e.g., the first, second, or third time window) exceeds a second predetermined threshold probability, the patient discharge date prediction system may automatically initiate time-specific discharge coordination workflows including scheduling patient transport within the particular time window, notifying bed management of anticipated bed availability within the particular time window, and coordinating pharmacy medication reconciliation for completion prior to the particular time window. In this way, the patient discharge date prediction system may use the calibrated discharge horizon probabilities and time-of-day discharge probabilities to trigger automated discharge preparation activities at a time when the predicted probability of discharge exceeds a configured threshold, thereby enabling earlier initiation of discharge logistics relative to a system that initiates discharge preparation only in response to a manual determination by a care provider or only in response to a current date falling within a predicted discharge date window.

At 1212, method 1200 includes updating the predictions as new data is received. Updating the predictions may include monitoring the plurality of data sources for new patient data, and in response to new patient data becoming available, repeating operations 1202 through 1210 to generate updated discharge horizon probabilities, updated time-of-day discharge probabilities, updated calibrated probabilities and confidence intervals, and updated user interface elements reflecting the updated predictions. For example, if new lab results, vital sign data, or operational workflow data are recorded for the patient, the patient discharge date prediction system may automatically re-execute operations 1202 through 1210 to generate updated predictions based on the new patient data. In various embodiments, the predictions may be updated at a predetermined frequency (e.g., every 15 minutes, every 30 minutes, every hour), in response to new patient data exceeding a threshold amount of new data, or in response to a user request to refresh the predictions. In some embodiments, updating the predictions at operation 1212 may include comparing the updated predictions with previous predictions to determine whether a change in the predictions exceeds a threshold change. If the change exceeds the threshold change, the patient discharge date prediction system may generate an alert or notification to one or more care providers of the patient indicating that the discharge timing predictions have changed. For example, if a patient previously predicted to be discharged on a current date with a 72% probability is now predicted to be discharged on the current date with a 30% probability, the patient discharge date prediction system may generate an alert indicating that the patient is no longer likely to be discharged on the current date. In this way, care providers may be notified of changes in discharge timing predictions that may affect discharge planning activities.

By performing method 1200, the patient discharge date prediction system may provide care providers with continuously updated, calibrated probabilistic discharge timing predictions that indicate not only when a patient is likely to be discharged but also how reliable those predictions are based on historical observed discharge rates. The care provider may use the calibrated probabilities and confidence intervals to prioritize discharge-related activities across a patient census, to coordinate staffing, transport, pharmacy, and post-acute placement services based on the relative likelihood of discharge occurring within specific time windows, and to assess a reliability of the predictions based on the width of the confidence intervals displayed in confidence interval display 1100 of FIG. 11. Referring now to FIG. 13, a flowchart of a method 1300 is shown for generating Wilson score interval confidence bounds for patient discharge probability predictions in accordance with the second embodiment. Method 1300 may be carried out by a patient discharge date prediction system, such as patient discharge date prediction system 900 of FIG. 9A, according to instructions stored in a non-transitory memory (e.g., non-transitory memory 206 of FIG. 2) of the patient discharge date prediction system, which may be executed by a processor (e.g., processor 204 of FIG. 2) of the patient discharge date prediction system. Method 1300 may be performed during a calibration phase of the patient discharge date prediction system, prior to or concurrently with deployment of patient LOS prediction model 902 for inference. In various embodiments, method 1300 may be performed periodically to update the Wilson score intervals based on newly accumulated historical discharge data, thereby maintaining a calibration accuracy of the confidence bounds over time as patient populations and discharge patterns change.

Method 1300 begins at 1302, where method 1300 includes binning patients by predicted probability bands. Binning patients by predicted probability bands may include retrieving historical discharge predictions generated by patient LOS prediction model 902 of FIG. 9A for a set of historical patients, and grouping the historical patients into a plurality of predicted probability bands based on the predicted probabilities generated for each historical patient. For example, the predicted probability bands may include a 0% to 10% band, a 10% to 20% band, a 20% to 30% band, a 30% to 40% band, a 40% to 50% band, a 50% to 60% band, a 60% to 70% band, a 70% to 80% band, an 80% to 90% band, and a 90% to 100% band. In various embodiments, the predicted probability bands may be of uniform width (e.g., 10 percentage points each), or the predicted probability bands may be of non-uniform width based on a distribution of predicted probabilities across the set of historical patients. For example, if a large number of historical patients have predicted probabilities between 60% and 80%, the 60% to 80% range may be divided into narrower bands (e.g., 60% to 65%, 65% to 70%, 70% to 75%, 75% to 80%) to increase a granularity of the calibration within that range.

In various embodiments, binning patients by predicted probability bands at operation 1302 may be performed separately for each probability category of discharge horizon probability distribution 930 and time-of-day probability distribution 932 of FIG. 9A. For example, a first set of predicted probability bands may be generated for the same-day discharge category of discharge horizon probability distribution 930; a second set of predicted probability bands may be generated for the next-day discharge category of discharge horizon probability distribution 930; a third set of predicted probability bands may be generated for the two-or-more-day discharge category of discharge horizon probability distribution 930; a fourth set of predicted probability bands may be generated for the first time window category of time-of-day probability distribution 932; a fifth set of predicted probability bands may be generated for the second time window category of time-of-day probability distribution 932; and a sixth set of predicted probability bands may be generated for the third time window category of time-of-day probability distribution 932. In this way, the calibration may be performed independently for each probability category, to account for differences in calibration characteristics across the different categories.

At 1304, method 1300 includes computing observed discharge rates within each band. Computing observed discharge rates within each band may include, for each predicted probability band generated at operation 1302, determining an actual discharge outcome for each historical patient within the predicted probability band, and computing a proportion of historical patients within the predicted probability band for whom the actual discharge outcome matched the predicted category. For example, for the 70% to 80% predicted probability band of the same-day discharge category, computing the observed discharge rate may include identifying all historical patients for whom patient LOS prediction model 902 predicted a same-day discharge probability between 70% and 80%, determining how many of those historical patients were actually discharged on the same day, and computing a ratio of the number of patients actually discharged on the same day to the total number of patients in the 70% to 80% predicted probability band. If 500 historical patients were in the 70% to 80% predicted probability band for same-day discharge, and 370 of those patients were actually discharged on the same day, the observed discharge rate for the 70% to 80% band would be 370 divided by 500, or 74%.

In various embodiments, computing the observed discharge rates at operation 1304 may include computing the observed discharge rate for each predicted probability band of each probability category (e.g., same-day, next-day, two-or-more-day, first time window, second time window, third time window). The observed discharge rates may indicate how well-calibrated the predicted probabilities of patient LOS prediction model 902 are for each predicted probability band. For example, if the observed discharge rate for the 70% to 80% predicted probability band is 74%, this indicates that the predicted probabilities in the 70% to 80% range are reasonably well-calibrated, as the observed rate (74%) falls within the predicted range (70% to 80%). Alternatively, if the observed discharge rate for the 70% to 80% predicted probability band were 55%, this would indicate that the predicted probabilities in the 70% to 80% range are poorly calibrated, as the model is overestimating the probability of discharge for patients in that band.

At 1306, method 1300 includes calculating Wilson score intervals based on the computed discharge rates. Calculating Wilson score intervals may include, for each predicted probability band, applying the Wilson score interval formula to the observed discharge rate computed at operation 1304 and a number of historical patients within the predicted probability band, to generate a lower confidence bound and an upper confidence bound around the observed discharge rate. The Wilson score interval may be calculated according to the following formula (for a 95% confidence level):

$$1 / ( 1 + {z}^{2} / n ) * ( \hat{p} + {z}^{2} / 2 n + z √ ( \hat{p} ( 1 - \hat{p} ) / n + {z}^{2} / 4 {n}^{2} ) )$$

$$where \hat{p} = observed discharge rate ,$$

$$n = number of patients in band ,$$

$$z = z - score for confidence level$$

The Wilson score interval may produce a lower confidence bound and an upper confidence bound that together form a confidence interval around the observed discharge rate. For example, for the 70% to 80% predicted probability band of the same-day discharge category, where the observed discharge rate is 74% and the number of historical patients in the band is 500, the Wilson score interval at a 95% confidence level may produce a lower confidence bound of 68% and an upper confidence bound of 79%. This indicates that, with 95% confidence, the true discharge rate for patients predicted to have a 70% to 80% probability of same-day discharge lies between 68% and 79%.

In various embodiments, the Wilson score interval may be selected over other confidence interval methods (e.g., the Wald interval or the Clopper-Pearson interval) because the Wilson score interval provides better coverage properties for proportions near 0 or 1, and for small sample sizes. The Wilson score interval does not produce confidence bounds that extend below 0% or above 100%, which may occur with the Wald interval for proportions near the boundaries. In this way, the Wilson score interval may provide more reliable confidence bounds for predicted probability bands at the extremes of the probability range.

At 1308, method 1300 includes storing the Wilson score intervals. Storing the Wilson score intervals may include storing in the memory, for each predicted probability band of each probability category, the observed discharge rate, the lower confidence bound, and the upper confidence bound in a data structure accessible to the patient discharge date prediction system during inference. The data structure may be stored in non-transitory memory 206 of FIG. 2, or in a database communicably coupled to the patient discharge date prediction system (e.g., computer systems and databases 230 of FIG. 2). In various embodiments, the data structure may be a lookup table indexed by probability category and predicted probability band, where each entry of the lookup table includes the observed discharge rate, the lower confidence bound, and the upper confidence bound for the corresponding predicted probability band and probability category.

For example, the lookup table may include an entry for the 70% to 80% predicted probability band of the same-day discharge category, where the entry includes an observed discharge rate of 74%, a lower confidence bound of 68%, and an upper confidence bound of 79%. The lookup table may include similar entries for each predicted probability band of each probability category, such that during inference, the patient discharge date prediction system may retrieve the appropriate Wilson score interval for any predicted probability generated by patient LOS prediction model 902.

At 1310, method 1300 includes generating confidence bounds during inference based on the stored intervals. Generating confidence bounds during inference may include, during operation of the patient discharge date prediction system to generate discharge predictions for current patients (e.g., as described in reference to method 1200 of FIG. 12), receiving a predicted probability from discharge horizon probability distribution 930 or time-of-day probability distribution 932 of FIG. 9A, identifying a predicted probability band corresponding to the predicted probability, retrieving the stored Wilson score interval (e.g., the observed discharge rate, the lower confidence bound, and the upper confidence bound) for the identified predicted probability band from the lookup table stored at operation 1308, and outputting the retrieved Wilson score interval as the calibrated probability and confidence interval for the predicted probability. The calibrated probability and confidence interval may then be provided to patient manager GUI display 952 of FIG. 9B for display in confidence interval display 960 of FIG. 9B, as described above in reference to FIGS. 9B, 10, and 11.

In various embodiments, generating confidence bounds during inference at operation 1310 may further include interpolating between adjacent predicted probability bands if the predicted probability falls near a boundary between two bands. For example, if the predicted probability is 70.5%, which is near the boundary between the 60% to 70% band and the 70% to 80% band, the patient discharge date prediction system may interpolate between the Wilson score intervals of the two adjacent bands to generate a more precise confidence interval. In other embodiments, the predicted probability may be assigned to the band within which it falls without interpolation.

By performing method 1300, the patient discharge date prediction system may generate statistically rigorous confidence bounds around discharge probability predictions that have been validated against historical observed discharge rates. The confidence bounds may provide care providers with a transparent indication of how reliable the discharge probability predictions are, based on how well-calibrated the predictions have been historically for patients in similar predicted probability bands. Further, by storing the Wilson score intervals in a lookup table and retrieving them during inference, the patient discharge date prediction system may generate the confidence bounds with minimal computational overhead during real-time inference, as the computationally intensive calibration calculations are performed during the calibration phase rather than during inference. In this way, method 1300 may enable the patient discharge date prediction system to provide calibrated confidence information to care providers without introducing latency into the real-time prediction pipeline.

FIG. 14A shows an LOS prediction model training system 1400 configured for training a patient LOS prediction model to generate multi-output predictions in accordance with the second embodiment described above in reference to FIGS. 9A-13. LOS prediction model training system 1400 may be similar to LOS prediction model training system 600 described above in reference to FIG. 6A, and elements of FIG. 14A having similar names to elements of FIG. 6A may be non-limiting examples of the similarly named elements of FIG. 6A. Elements of FIG. 14A that have been described above in reference to FIG. 6A are not described again here in detail, except to the extent that they differ from or extend the functionality described in reference to FIG. 6A.

In FIG. 14A, historical patient data 614 may be received at a dataset generator 616 within a training module 1402. Training module 1402 may be a non-limiting example of training module 210 of FIG. 2. Dataset generator 616 may process historical patient data 614 to generate training pairs 1404 and test pairs 1410. In contrast to the training pairs 608 and test pairs 618 described above in reference to FIG. 6A, which each include a data vector 610 and an actual LOS 612, training pairs 1404 and test pairs 1410 of FIG. 14A may each include four components: a data vector 610, an actual LOS 612, an actual discharge horizon category 1406, and an actual time-of-day discharge window category 1408.

Data vector 610 may be a vector of values encoding patient data of a historical patient, as described above in reference to data vector 552 of FIG. 5B. Actual LOS 612 may be the actual length of stay of the historical patient corresponding to data vector 610, as described above in reference to FIG. 6A, which may be used as a first ground truth for training the patient LOS prediction model 602.

Actual discharge horizon category 1406 may be a categorical label indicating a discharge horizon of the historical patient relative to a reference date within the historical patient data, as a second ground truth for training the patient LOS prediction model 602. In various embodiments, actual discharge horizon category 1406 may be one of three categories: a first category indicating that the historical patient was discharged on the same day as the reference date (e.g., “today”), a second category indicating that the historical patient was discharged on the day following the reference date (e.g., “tomorrow”), or a third category indicating that the historical patient was discharged two or more days after the reference date (e.g., “2+ days”). The reference date may be a date at which the data vector 610 was generated from the historical patient data, such that actual discharge horizon category 1406 reflects how many days remained between the reference date and the actual discharge date of the historical patient. In this way, actual discharge horizon category 1406 may serve as a ground truth label for training patient LOS prediction model 602 to predict a discharge horizon probability distribution, as described above in reference to discharge horizon probability distribution 930 of FIG. 9A.

Actual time-of-day discharge window category 1408 may be a categorical label indicating a time-of-day window within which the historical patient was actually discharged, as a third ground truth for training the patient LOS prediction model 602. In various embodiments, actual time-of-day discharge window category 1408 may be one of three categories: a first category indicating that the historical patient was discharged within a first time window (e.g., 12 AM to 11 AM), a second category indicating that the historical patient was discharged within a second time window (e.g., 12 PM to 6 PM), or a third category indicating that the historical patient was discharged within a third time window (e.g., 7 PM to 11 PM). Actual time-of-day discharge window category 1408 may be derived from a timestamp of a discharge event recorded in the historical patient data. In this way, actual time-of-day discharge window category 1408 may serve as a ground truth label for training patient LOS prediction model 602 to predict a time-of-day probability distribution, as described above in reference to time-of-day probability distribution 932 of FIG. 9A.

Dataset generator 616 may generate actual discharge horizon category 1406 and actual time-of-day discharge window category 1408 for each historical patient by examining a discharge date and discharge time recorded in historical patient data 614 for the historical patient. For example, dataset generator 616 may compare the discharge date of the historical patient to the reference date to determine actual discharge horizon category 1406, and may examine the discharge time of the historical patient to determine actual time-of-day discharge window category 1408. In various embodiments, dataset generator 616 may generate multiple training pairs 1404 for a single historical patient by selecting multiple reference dates during the historical patient's stay. For example, a first training pair may be generated with a first reference date that is three days prior to the actual discharge date, resulting in an actual discharge horizon category 1406 of “2+ days”; a second training pair may be generated with a second reference date that is one day prior to the actual discharge date, resulting in an actual discharge horizon category 1406 of “next day”; and a third training pair may be generated with a third reference date that is the same day as the actual discharge date, resulting in an actual discharge horizon category 1406 of “current date.” In this way, dataset generator 616 may generate training data that captures the progression of discharge horizon categories over the course of a patient's stay, enabling patient LOS prediction model 602 to learn how discharge horizon probabilities change as a patient approaches discharge.

Training pairs 1404 may be provided to patient LOS prediction model 602 within neural network module 604 for training. Patient LOS prediction model 602 may be configured with multiple output branches to simultaneously predict actual LOS 612, actual discharge horizon category 1406, and actual time-of-day discharge window category 1408 from data vector 610. In various embodiments, patient LOS prediction model 602 may include a shared feature extraction portion comprising one or more convolutional layers and pooling layers, as described above in reference to FIG. 7, followed by multiple output branches. A first output branch may include one or more fully connected layers configured to output a predicted LOS value. A second output branch may include one or more fully connected layers followed by a softmax activation layer configured to output a probability distribution across the three discharge horizon categories. A third output branch may include one or more fully connected layers followed by a softmax activation layer configured to output a probability distribution across the three time-of-day discharge window categories.

During training, patient LOS prediction model 602 may minimize a combined loss function that includes a first loss component measuring a difference between the predicted LOS and actual LOS 612, a second loss component measuring a difference between the predicted discharge horizon probability distribution and actual discharge horizon category 1406, and a third loss component measuring a difference between the predicted time-of-day probability distribution and actual time-of-day discharge window category 1408. In various embodiments, the first loss component may be a mean squared error loss or a mean absolute error loss, and the second and third loss components may each be a cross-entropy loss. The combined loss function may be a weighted sum of the first, second, and third loss components, where the weights may be configured to balance the relative importance of each output during training. By training patient LOS prediction model 602 using the combined loss function, the shared feature extraction portion of patient LOS prediction model 602 may learn representations that are informative for all three prediction tasks simultaneously, which may increase a generalization performance of patient LOS prediction model 602 relative to training separate models for each prediction task independently.

Once the training pairs 1404 have been generated, a portion of the training pairs 1404 may be assigned to a test dataset, as test pairs 1410. Test pairs 1410 may be used to prevent overfitting, as described above in reference to test pairs 618 of FIG. 6A.

LOS prediction model training system 1400 may include a validator 620. Validator 620 may validate a performance of patient LOS prediction model 602 on test pairs 1410 by evaluating the accuracy of patient LOS prediction model 602 on each of the three output tasks. For example, validator 620 may evaluate an error rate of the predicted LOS relative to actual LOS 612, a classification accuracy of the predicted discharge horizon category relative to actual discharge horizon category 1406, and a classification accuracy of the predicted time-of-day discharge window category relative to actual time-of-day discharge window category 1408. If the performance of patient LOS prediction model 602 on test pairs 1410 meets one or more threshold accuracy criteria for each of the three output tasks, validator 620 may output a trained patient LOS prediction model 622 for deployment in the inference pipeline described below in reference to FIG. 14B.

In various embodiments, patient LOS prediction model 602 may be configured with fewer than three output branches depending on a configuration of the patient discharge date prediction system. For example, in some embodiments, patient LOS prediction model 602 may be configured with a first output branch for predicting LOS and a second output branch for predicting discharge horizon category 1452, without including a third output branch for predicting time-of-day window category 1454. In such embodiments, training pairs 1404 and test pairs 1410 may each include a data vector 610, an actual LOS 612, and an actual discharge horizon category 1406, without including an actual time-of-day discharge window category 1408, and the combined loss function used during training may include only the first loss component measuring a difference between the predicted LOS and actual LOS 612 and the second loss component measuring a difference between the predicted discharge horizon probability distribution and actual discharge horizon category 1406. Similarly, in other embodiments, patient LOS prediction model 602 may be configured with a first output branch for predicting LOS and a third output branch for predicting time-of-day window category 1454, without including a second output branch for predicting discharge horizon category 1452. In such embodiments, training pairs 1404 and test pairs 1410 may each include a data vector 610, an actual LOS 612, and an actual time-of-day discharge window category 1408, without including an actual discharge horizon category 1406, and the combined loss function used during training may include only the first loss component measuring a difference between the predicted LOS and actual LOS 612 and the third loss component measuring a difference between the predicted time-of-day probability distribution and actual time-of-day discharge window category 1408. In this way, patient LOS prediction model 602 may be trained to generate only the discharge horizon probability distribution, only the time-of-day discharge probability distribution, or both distributions, depending on the operational demands of the healthcare facility.

Referring now to FIG. 14B, a deployment diagram 1450 of trained patient LOS prediction model 622 of FIG. 14A is shown in use during deployment within a patient discharge date prediction system 654. Patient discharge date prediction system 654 may be a non-limiting example of patient discharge date prediction system 100 of FIG. 1. Deployment diagram 1450 may be similar to deployment diagram 650 of FIG. 6B, and elements of FIG. 14B having similar names to elements of FIG. 6B may be non-limiting examples of the similarly named elements of FIG. 6B. In contrast to FIG. 6B, where validated patient LOS prediction model 622 outputs a single LOS prediction 662 for each patient 658, in FIG. 14B, validated patient LOS prediction model 622 outputs three predictions simultaneously for each patient 658.

During operation of patient discharge date prediction system 654, a set of current patients 656 may be selected for generating discharge date predictions, as described above in reference to FIG. 6B. The set of current patients 656 includes a plurality of individual patients 658, where each patient 658 is represented by a data vector 660. Data vector 660 may be generated from current patient data of patient 658 in the manner described above in reference to FIG. 5B, and may additionally include operational workflow data 922 as described above in reference to FIG. 9A.

For each patient 658 of the set of current patients 656, a corresponding data vector 660 may be inputted into validated patient LOS prediction model 622 within inference module 652. Inference module 652 may be a non-limiting example of inference module 212 of FIG. 2. In a single forward pass of data vector 660 through validated patient LOS prediction model 622, inference module 652 may generate three outputs for patient 658: an LOS prediction 662, a discharge horizon category 1452, and a time-of-day window category 1454.

LOS prediction 662 may be a predicted number of days that patient 658 is expected to remain a patient of the healthcare facility, as described above in reference to FIG. 6B. LOS prediction 662 may be provided to a date calculator (e.g., date calculator 906 of FIG. 9A) to generate a predicted discharge date for patient 658.

Discharge horizon category 1452 may be a probability distribution across the three discharge horizon categories (e.g., on a current date, on the day after the current date, two or more days after the current date), as generated by the softmax output layer of the second output branch of validated patient LOS prediction model 622. Discharge horizon category 1452 may correspond to discharge horizon probability distribution 930 described above in reference to FIG. 9A, and may be provided to Wilson score interval calibration 934 of FIG. 9A to generate calibrated horizon probabilities and confidence intervals 940.

Time-of-day window category 1454 may be a probability distribution across the three time-of-day discharge window categories (e.g., morning, afternoon, and evening), as generated by the softmax output layer of the third output branch of validated patient LOS prediction model 622. Time-of-day window category 1454 may correspond to time-of-day probability distribution 932 described above in reference to FIG. 9A, and may be provided to Wilson score interval calibration 934 of FIG. 9A to generate calibrated time-of-day probabilities and confidence intervals 942.

By generating LOS prediction 662, discharge horizon category 1452, and time-of-day window category 1454 simultaneously in a single forward pass through validated patient LOS prediction model 622, inference module 652 may generate all three predictions for each patient 658 without requiring multiple separate model evaluations. This may reduce a computational cost of generating the predictions relative to deploying three separate models, and may further increase a consistency among the three predictions by virtue of the shared feature extraction portion of validated patient LOS prediction model 622 producing a unified internal representation of the patient data from which all three outputs are derived. The stacked representation of current patients 656 and the corresponding stacked output representation in FIG. 14B indicate that inference module 652 may process data vectors 660 of multiple patients 658 in a batch, generating LOS prediction 662, discharge horizon category 1452, and time-of-day window category 1454 for each patient 658 of the batch in a single batch inference operation.

Correspondingly, during deployment within patient discharge date prediction system 654, validated patient LOS prediction model 622 within inference module 652 may generate fewer than three outputs for each patient 658 depending on the configuration of patient LOS prediction model 602. For example, in embodiments where patient LOS prediction model 602 was trained with only the first output branch and the second output branch, validated patient LOS prediction model 622 may output LOS prediction 662 and discharge horizon category 1452 for each patient 658, without outputting time-of-day window category 1454. In embodiments where patient LOS prediction model 602 was trained with only the first output branch and the third output branch, validated patient LOS prediction model 622 may output LOS prediction 662 and time-of-day window category 1454 for each patient 658, without outputting discharge horizon category 1452. In embodiments where patient LOS prediction model 602 was trained with all three output branches, validated patient LOS prediction model 622 may output LOS prediction 662, discharge horizon category 1452, and time-of-day window category 1454 simultaneously for each patient 658 in a single forward pass, as described above. The selection of which output branches to include in patient LOS prediction model 602 may be determined during a configuration phase of the patient discharge date prediction system based on the operational requirements of the healthcare facility, and may be updated by retraining patient LOS prediction model 602 with a different set of output branches if the operational requirements change.

Thus, the systems, methods, and graphical user interfaces provided herein may increase the accuracy and timeliness of patient discharges for high-demand, high-resource hospitals and/or hospital units. By establishing a system that automatically receives data from all EHRs in real time or near real time, aggregates that information regardless of data format, and periodically updates a GUI as data is received, the approach of the disclosure allows care providers to make informed decisions based on when patients may be discharged, thereby improving patient care.

In contrast, in prior systems when a care provider attempted to determine when a patient would be ready to discharge, errors could be made due to delays in individual care providers obtaining all desired parameters for accurately predicting patient discharge dates. For example, a patient's condition may have improved to the point where the patient would be ready for a discharge, but limited care provider resources may result in the patient being kept in the hospital for an additional 12, 24, or more hours, thereby utilizing hospital resources unnecessarily. The methods and systems solve this problem by providing specific, dynamically updating patient discharge window predictions, which can be selectively adjusted to indicate a greater or lesser degree of confidence in the discharge date prediction system's accuracy. In this way, the approach described herein provides an improvement to the capability of the healthcare system as a whole. The disclosure provides a specific way of improving the capability of the healthcare system, by dynamically updating patient discharge window prediction lists. The disclosure further provides a specific improvement to the way computers operate by aggregating EHR data for multiple patients in one location and updating the patient discharge window predictions in real-time, which may obviate the demand for users to have to navigate through multiple different data files, manually update information as availability changes, and so forth, thereby increasing the efficiency of the operation of the computer for the user.

Further, hospitals may not be configured to share patient discharge data in real-time, across different units of the hospital. Thus, prior methods of discharge readiness determination demanded manual collection of data (e.g., reading patient charts in person, discussing patient status with other care providers, visually inspecting each room for bed occupancy) and aggregation in a spreadsheet or other document, with updates also made manually. Systems were not available to automatically pull EHR data from multiple patients in multiple hospital units and then aggregate the pulled data into a visually clear format where healthcare decisions can be quickly made just by glancing at a GUI, as described in the embodiments herein.

The GUIs described herein provide a specific manner of displaying a limited set of information to a user (patient discharge date windows), rather than using conventional user interface methods to display a generic index on a computer, requiring the user to step through many layers of menu options to reach the desired data, or burying the desired data within all hospital data. Thus, the user experience with the computer may be improved and made more efficient.

Furthermore, by displaying a limited set of information via the discharge window predictions as described herein, operation of the computing device(s) that collect and render the data for display may be improved by reducing the processing demands of the computing device(s), thereby increasing the efficiency of the computing device(s). Further, in some examples the data is processed in real time and updates to the GUIs are made continuously as data is received, and therefore undue processing lags that may occur if updates were made at predefined discrete time points may be reduced, which may increase the efficiency of the computing device(s).

Via the disclosed patient discharge date prediction system, discharge window predictions for a patient may be displayed in a manner that is easy to visually parse and act on in a reduced amount of time. The discharge window predictions may be displayed via small graphical elements with minimal text, which may allow a large number of discharge window predictions to be included on the same screen. The patient information may be stored in different databases that would otherwise be accessed via individual interfaces, and by using the patient discharge date prediction system to aggregate the patient information, an amount of time taken to review relevant patient information to predict a discharge date of a patient may be reduced. The technical effect of automatically generating discharge window predictions for patients of a hospital based on patient data using a length-of-stay prediction model is that resources of the hospital may be more efficiently managed to meet demands. By consolidating patient data from a plurality of disparate data sources into a single data vector and processing the data vector through the patient LOS prediction model in a single forward pass, patient discharge date prediction system 100 may reduce a number of separate database queries, network transactions, and data retrieval operations that would otherwise be performed by individual care providers accessing individual EHR systems, computer systems, and databases independently. Rather than each care provider of a healthcare facility independently querying an EHR database, computer systems and databases, and patient monitoring devices to retrieve patient data for each patient under the care provider's supervision, the disclosed patient discharge date prediction system may aggregate the patient data from the plurality of data sources in a single batch retrieval operation, encode the patient data into data vectors for a set of current patients, and generate LOS predictions for the set of current patients in a single batch inference operation through the patient LOS prediction model. This batch processing approach may reduce a total number of network communications between the patient discharge date prediction system and the plurality of data sources, thereby reducing network bandwidth consumption and processing overhead on the EHR database and computer systems and databases relative to a scenario in which each care provider independently retrieves and analyzes patient data from the plurality of data sources.

Further, by generating and caching the discharge window predictions at predetermined intervals or in response to new patient data exceeding a threshold amount of new data, the disclosed patient discharge date prediction system may reduce redundant computation that would otherwise occur if multiple care providers each independently requested discharge predictions for overlapping sets of patients. For example, if a first care provider and a second care provider each oversee a partially overlapping set of patients, the patient discharge date prediction system may generate a single set of discharge window predictions for all patients of the healthcare facility, and serve the relevant subset of predictions to each care provider via the UI without re-executing the inference pipeline for each individual request. Additionally, by processing the patient data in background operations and updating the predictions asynchronously as new data becomes available via the secure feeds, the patient discharge date prediction system may distribute computational load over time rather than concentrating processing demands at discrete time points when care providers simultaneously access the system. This asynchronous processing approach may reduce peak computational load on processors and reduce latency experienced by care providers when viewing the discharge window predictions in the UI, as the predictions may be pre-computed and stored in non-transitory memory prior to the care provider requesting display of the predictions. In this way, the patient discharge date prediction system may increase an operational efficiency of the computing infrastructure of the healthcare facility by reducing redundant data retrieval operations, distributing computational load, and minimizing network traffic between the patient discharge date prediction system and the plurality of data sources from which patient data is retrieved.

The disclosure also provides support for a method for a patient management system of a healthcare facility, comprising: receiving, via secure feeds from a plurality of electronic health record databases, patient data, wherein the patient data comprises temporal medical data in a plurality of different formats, receiving operational workflow data from one or more operational systems of the healthcare facility, encoding the received patient data and the received operational workflow data into data vectors, predicting, based on the encoded data vectors, using a trained length of stay (LOS) prediction model, a date for discharging a patient of the healthcare facility, and a discharge horizon probability distribution indicating a first probability that the patient will be discharged on a specified date, a second probability that the patient will be discharged the day after the specified date, and a third probability that the patient will be discharged two or more days from a specified date, wherein the LOS prediction model is trained on historical patient data of the healthcare facility, and wherein the LOS prediction model is a convolution neural network (CNN), and wherein the LOS prediction model is trained by: dividing the historical patient data into a plurality of groupings based on length of stay ranges, classifying each patient of the historical patient data into one of the plurality of groupings, generating data vectors for each patient, each data vector encoding patient data of the respective patient, training the LOS prediction model sequentially on each grouping using the data vectors of patients in that grouping and actual length of stay values, determining an accuracy rating for each grouping based on prediction performance of the LOS prediction model for that grouping, reordering the plurality of groupings based on the accuracy ratings, and retraining the LOS prediction model on the reordered groupings, and displaying the predicted date for discharging the patient and the discharge horizon probability distribution in a user interface (UI) on a display device of the patient management system. In a first example of the method, training the LOS prediction model sequentially on each grouping further comprises training the LOS prediction model using a combined loss function that includes a first loss component for LOS, a second loss component (cross-entropy) for a discharge horizon output, and a third loss component (cross-entropy) for a time-of-day output. In a second example of the method, optionally including the first example, training the LOS prediction model sequentially on each grouping further comprises generating multiple training pairs for a single historical patient by selecting multiple reference dates during a stay of the patient, resulting in different actual discharge horizon categories for the same patient. In a third example of the method, optionally including one or both of the first and second examples, the operational workflow data includes: rounding schedule data indicating times at which attending physicians conduct patient rounds on a given unit, order entry timing data from one or more computerized physician order entry (CPOE) systems of the healthcare facility, consult completion timing data indicating average durations and completion times of pending consults that may represent barriers to discharge, placement logistics data indicating lead times and scheduling constraints associated with post-acute placement services, and transport scheduling data indicating availability windows and scheduling patterns of patient transport services. In a fourth example of the method, optionally including one or more or each of the first through third examples, the method further comprises: predicting, based on the encoded data vectors and using the trained LOS prediction model, a time-of-day discharge probability distribution indicating a fourth probability that the patient will be discharged within a first time window, a fifth probability that the patient will be discharged within a second time window, and a sixth probability that the patient will be discharged within a third time window, and displaying the predicted date for discharging the patient, the discharge horizon probability distribution, and the time-of-day discharge probability distribution in the UI. In a fifth example of the method, optionally including one or more or each of the first through fourth examples, the method further comprises: updating the patient data asynchronously, periodically predicting the date for discharging the patient, the discharge horizon probability distribution, and the time-of-day discharge probability distribution, and updating the UI with the updated discharge date, the discharge horizon probability distribution, and the time-of-day discharge probability distribution. In a sixth example of the method, optionally including one or more or each of the first through fifth examples, displaying the predicted date for discharging the patient, the discharge horizon probability distribution, and the time-of-day discharge probability distribution in the UI further comprises displaying the predicted date for discharging the patient, the discharge horizon probability distribution, and the time-of-day discharge probability distribution in the UI alongside other data of the patient and discharge display elements in an integrated view of the UI, the integrated view including clinical information of a plurality of patients of the healthcare facility. In a seventh example of the method, optionally including one or more or each of the first through sixth examples, one or more of the discharge horizon probability distribution and the time-of-day discharge probability distribution are displayed in a popup display panel in response to a user selecting an element of the UI. In a eighth example of the method, optionally including one or more or each of the first through seventh examples, the method further comprises: in response to determining that a probability of the discharge horizon probability distribution indicating that the patient will be discharged on a current date exceeds a predetermined threshold probability, automatically initiating one or more discharge preparation workflows for the patient, the one or more discharge preparation workflows including one or more of: generating a notification to one or more care providers of the patient indicating one or more administrative tasks to be performed prior to discharging the patient, arranging transportation for the patient, initiating paperwork associated with discharging the patient, and generating a message to coordinate staffing, pharmacy, or post-acute placement services based on the time-of-day discharge probability distribution. In a ninth example of the method, optionally including one or more or each of the first through eighth examples, the method further comprises: in response to determining that a probability of the time-of-day discharge probability distribution indicating that the patient will be discharged within the first time window exceeds a second predetermined threshold probability, automatically initiating time-specific discharge coordination workflows including one or more of: scheduling patient transport within the morning time window, notifying bed management of anticipated bed availability within the first time window, and coordinating pharmacy medication reconciliation for completion prior to the first time window. In a tenth example of the method, optionally including one or more or each of the first through ninth examples, the method further comprises: applying Wilson score interval calibration to the discharge horizon probability distribution and the time-of-day discharge probability distribution based on historical observed discharge rates of the healthcare facility to generate calibrated probabilities and associated confidence intervals for each probability of the discharge horizon probability distribution and the time-of-day discharge probability distribution, wherein applying Wilson score interval calibration comprises: binning historical patients into a plurality of predicted probability bands based on predicted probabilities generated by the trained LOS prediction model for the historical patients, computing an observed discharge rate within each predicted probability band based on actual discharge outcomes of the historical patients within the predicted probability band, calculating a Wilson score interval for each predicted probability band based on the observed discharge rate and a number of historical patients within the predicted probability band, the Wilson score interval including a lower confidence bound and an upper confidence bound, storing the Wilson score interval for each predicted probability band in a data structure accessible to the patient management system during inference, and displaying the calibrated probabilities and the associated confidence intervals in the UI. In a eleventh example of the method, optionally including one or more or each of the first through tenth examples, displaying the calibrated probabilities and the associated confidence intervals in the UI comprises: displaying a confidence interval display element including a point estimate representing an observed discharge rate for a predicted probability band corresponding to a selected probability, a lower confidence bound derived from the Wilson score interval for the predicted probability band, an upper confidence bound derived from the Wilson score interval for the predicted probability band, and a graphical confidence range indicator depicting a span between the lower confidence bound and the upper confidence bound with the point estimate indicated at a position within the span.

The disclosure also provides support for a method for a patient discharge date prediction system, comprising: receiving, at the patient discharge date prediction system, secure feeds from one or more electronic health record databases of a hospital network, receiving operational workflow data from one or more operational systems of a healthcare facility, encoding the received patient data and the received operational workflow data into data vectors, receiving a selected confidence level from a user, predicting, for each patient of a first set of patients associated with the healthcare facility, a predicted discharge date and a discharge horizon probability distribution indicating a first probability that the patient will be discharged on a current date, a second probability that the patient will be discharged on a day after the current date, and a third probability that the patient will be discharged two or more days from the current date using a trained length of stay (LOS) prediction model based on the encoded data vectors, the trained LOS prediction model trained based on historical patient data, predicting, for each patient of the first set of patients, a predicted discharge date window based on the trained LOS prediction model, the encoded data vectors, and the selected confidence level, displaying a graphical user interface (GUI) that displays, for each patient of the first set of patients, the predicted discharge date, the predicted discharge date window of the patient, and the discharge horizon probability distribution, and wherein training the LOS prediction model comprises: dividing the historical patient data into a plurality of groupings based on length of stay ranges, classifying each patient of the historical patient data into one of the plurality of groupings, generating data vectors for each patient, each data vector encoding patient data of the respective patient, training the LOS prediction model sequentially on each grouping using the data vectors of patients in that grouping and actual length of stay values, determining an accuracy rating for each grouping based on prediction performance of the LOS prediction model for that grouping, reordering the plurality of groupings based on the accuracy ratings, and retraining the LOS prediction model on the reordered groupings, and wherein a width of each of the predicted discharge date window is at least partially based on the accuracy ratings. In a first example of the method, the method further comprises: predicting, based on the encoded data vectors and using the trained LOS prediction model, a time-of-day discharge probability distribution indicating a fourth probability that the patient will be discharged within a first time window, a fifth probability that the patient will be discharged within a second time window, and a sixth probability that the patient will be discharged within a third time window, and displaying the predicted date for discharging the patient, the discharge horizon probability distribution, and the time-of-day discharge probability distribution in the GUI.

The disclosure also provides support for a patient discharge date prediction system, comprising: one or more processors, and memory storing instructions executable by the one or more processors to: receive, at the patient discharge date prediction system, a secure feed from one or more electronic health record databases of a hospital network, the secure feed including patient data of a plurality of patients, receive operational workflow data from one or more operational systems of the hospital, output, for display on a display device, a graphical user interface (GUI) that includes a discharge date and a discharge horizon probability distribution of each patient of the plurality of patients predicted by a length of stay (LOS) prediction model based on an encoding of both of the patient data of the patient included in the secure feed and the operational workflow data, the LOS prediction model trained on de-identified historical patient data of the one or more electronic health record databases, wherein the discharge horizon probability distribution indicates a first probability that the patient will be discharged on a current date, a second probability that the patient will be discharged the day after the current date, and a third probability that the patient will be discharged in two or more days after the current date, and wherein the LOS prediction model is a convolution neural network (CNN), and the LOS prediction model is trained by: dividing the de-identified historical patient data into a plurality of groupings based on length of stay ranges, classifying each patient of the de-identified historical patient data into one of the plurality of groupings, generating data vectors for each patient, each data vector encoding patient data of the respective patient, training the LOS prediction model sequentially on each grouping using the data vectors of patients in that grouping and actual length of stay values, determining an accuracy rating for each grouping based on prediction performance of the LOS prediction model for that grouping, reordering the plurality of groupings based on the accuracy ratings, and retraining the LOS prediction model on the reordered groupings. In a first example of the system, further instructions are stored in the memory that when executed, cause the one or more processors to: receive a selected confidence level from a user, predict, for each patient of the plurality of patients, a discharge date window based on the trained LOS prediction model, the encoded data vectors, and the selected confidence level, and display the discharge date window in the GUI, wherein the predicted discharge date window is a window of time greater than a calendar day. In a second example of the system, optionally including the first example, further instructions are stored in the memory that when executed, cause the one or more processors to: apply Wilson score interval calibration to the discharge horizon probability distribution based on historical observed discharge rates of the hospital network to generate calibrated probabilities and associated confidence intervals for each probability of the discharge horizon probability distribution, wherein applying Wilson score interval calibration comprises: binning historical patients into a plurality of predicted probability bands based on predicted probabilities generated by the LOS prediction model for the historical patients, computing an observed discharge rate within each predicted probability band based on actual discharge outcomes of the historical patients within the predicted probability band, calculating a Wilson score interval for each predicted probability band based on the observed discharge rate and a number of historical patients within the predicted probability band, the Wilson score interval including a lower confidence bound and an upper confidence bound, and storing the Wilson score interval for each predicted probability band in a data structure accessible to the patient discharge date prediction system during inference, and display the calibrated probabilities and the associated confidence intervals in the GUI. In a third example of the system, optionally including one or both of the first and second examples, the data structure is a lookup table, and wherein further instructions are stored in the memory that when executed, cause the one or more processors to periodically recalibrate the Wilson score intervals by: accumulating additional historical discharge data subsequent to a previous calibration, re-binning historical patients including patients associated with the additional historical discharge data into the plurality of predicted probability bands, recomputing the observed discharge rate within each predicted probability band based on actual discharge outcomes of the historical patients including the patients associated with the additional historical discharge data, recalculating the Wilson score interval for each predicted probability band based on the recomputed observed discharge rate and an updated number of historical patients within the predicted probability band, and replacing previously stored Wilson score intervals in the lookup table with the recalculated Wilson score intervals. In a fourth example of the system, optionally including one or more or each of the first through third examples, further instructions are stored in the memory that when executed, cause the one or more processors to: predict, based on the encoding of both of the patient data and the operational workflow data, using the LOS prediction model, a time-of-day discharge probability distribution indicating a fourth probability that the patient will be discharged within a first time window, a fifth probability that the patient will be discharged within a second time window, and a sixth probability that the patient will be discharged within a third time window, wherein the LOS prediction model generates the discharge date, the discharge horizon probability distribution, and the time-of-day discharge probability distribution simultaneously during a single forward pass of the encoding through the CNN, and display the time-of-day discharge probability distribution in the GUI. In a fifth example of the system, optionally including one or more or each of the first through fourth examples, further instructions are stored in the memory that when executed, cause the one or more processors to: in response to determining that the first probability of the discharge horizon probability distribution indicating that the patient will be discharged on the current date exceeds a predetermined threshold probability, automatically initiate one or more discharge preparation workflows for the patient, the one or more discharge preparation workflows including one or more of: generating a notification to one or more care providers of the patient indicating one or more administrative tasks to be performed prior to discharging the patient, arranging transportation for the patient, initiating paperwork associated with discharging the patient, and generating a message to coordinate staffing, pharmacy, or post-acute placement services based on the time-of-day discharge probability distribution, and in response to determining that a probability of the time-of-day discharge probability distribution indicating that the patient will be discharged within the first time window exceeds a second predetermined threshold probability, automatically initiate time-specific discharge coordination workflows including one or more of: scheduling patient transport within the first time window, notifying bed management of anticipated bed availability within the first time window, and coordinating pharmacy medication reconciliation for completion prior to the first time window.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a patient management system of a healthcare facility, comprising:
receiving, via secure feeds from a plurality of electronic health record databases, patient data, wherein the patient data comprises temporal medical data in a plurality of different formats;
receiving operational workflow data from one or more operational systems of the healthcare facility;
encoding the received patient data and the received operational workflow data into data vectors;
predicting, based on the encoded data vectors, using a trained length of stay (LOS) prediction model, a date for discharging a patient of the healthcare facility, and a discharge horizon probability distribution over possible discharge times;
wherein the LOS prediction model is trained on historical patient data of the healthcare facility; and
wherein the LOS prediction model is a convolution neural network (CNN), and wherein the LOS prediction model is trained by: dividing the historical patient data into a plurality of groupings based on length of stay ranges; classifying each patient of the historical patient data into one of the plurality of groupings; generating data vectors for each patient, each data vector encoding patient data of the respective patient; training the LOS prediction model sequentially on each grouping using the data vectors of patients in that grouping and actual length of stay values; determining an accuracy rating for each grouping based on prediction performance of the LOS prediction model for that grouping;
reordering the plurality of groupings based on the accuracy ratings; and retraining the LOS prediction model on the reordered groupings; and
displaying the predicted date for discharging the patient and the discharge horizon probability distribution in a user interface (UI) on a display device of the patient management system.

2. The method of claim 1, wherein training the LOS prediction model sequentially on each grouping further comprises training the LOS prediction model using a combined loss function that includes a first loss component for LOS, a second loss component (cross-entropy) for a discharge horizon output, and a third loss component (cross-entropy) for a time-of-day output.

3. The method of claim 1, wherein training the LOS prediction model sequentially on each grouping further comprises generating multiple training pairs for a single historical patient by selecting multiple reference dates during a stay of the patient, resulting in different actual discharge horizon categories for the same patient.

4. The method of claim 1, wherein the operational workflow data includes:
rounding schedule data indicating times at which attending physicians conduct patient rounds on a given unit;
order entry timing data from one or more computerized physician order entry (CPOE) systems of the healthcare facility;
consult completion timing data indicating average durations and completion times of pending consults that may represent barriers to discharge;
placement logistics data indicating lead times and scheduling constraints associated with post-acute placement services; and
transport scheduling data indicating availability windows and scheduling patterns of patient transport services.

5. The method of claim 1, further comprising:
predicting, based on the encoded data vectors and using the trained LOS prediction model, a time-of-day discharge probability distribution indicating a fourth probability that the patient will be discharged within a first time window of a discharge day, a fifth probability that the patient will be discharged within a second time window of the discharge day, and a sixth probability that the patient will be discharged within a third time window of the discharge day; and
displaying the predicted date for discharging the patient, the discharge horizon probability distribution, and the time-of-day discharge probability distribution in the UI.

6. The method of claim 5, further comprising:
updating the patient data asynchronously;
periodically predicting the date for discharging the patient, the discharge horizon probability distribution, and the time-of-day discharge probability distribution; and
updating the UI with the updated discharge date, the discharge horizon probability distribution, and the time-of-day discharge probability distribution.

7. The method of claim 5, wherein displaying the predicted date for discharging the patient, the discharge horizon probability distribution, and the time-of-day discharge probability distribution in the UI further comprises displaying the predicted date for discharging the patient, the discharge horizon probability distribution, and the time-of-day discharge probability distribution in the UI alongside other data of the patient and discharge display elements in an integrated view of the UI, the integrated view including clinical information of a plurality of patients of the healthcare facility.

8. The method of claim 7, wherein one or more of the discharge horizon probability distribution and the time-of-day discharge probability distribution are displayed in a popup display panel in response to a user selecting an element of the UI.

9. The method of claim 5, further comprising:
in response to determining that a probability of the discharge horizon probability distribution indicating that the patient will be discharged on a current date exceeds a predetermined threshold probability, automatically initiating one or more discharge preparation workflows for the patient, the one or more discharge preparation workflows including one or more of:
generating a notification to one or more care providers of the patient indicating one or more administrative tasks to be performed prior to discharging the patient;
arranging transportation for the patient;
initiating paperwork associated with discharging the patient; and
generating a message to coordinate staffing, pharmacy, or post-acute placement services based on the time-of-day discharge probability distribution.

10. The method of claim 5, further comprising:
in response to determining that a probability of the time-of-day discharge probability distribution indicating that the patient will be discharged within the first time window exceeds a second predetermined threshold probability, automatically initiating time-specific discharge coordination workflows including one or more of:
scheduling patient transport within the first time window;
notifying bed management of anticipated bed availability within the first time window; and
coordinating pharmacy medication reconciliation for completion prior to the first time window.

11. The method of claim 5, further comprising:
applying Wilson score interval calibration to the discharge horizon probability distribution and the time-of-day discharge probability distribution based on historical observed discharge rates of the healthcare facility to generate calibrated probabilities and associated confidence intervals for each probability of the discharge horizon probability distribution and the time-of-day discharge probability distribution, wherein applying Wilson score interval calibration comprises:
binning historical patients into a plurality of predicted probability bands based on predicted probabilities generated by the trained LOS prediction model for the historical patients;
computing an observed discharge rate within each predicted probability band based on actual discharge outcomes of the historical patients within the predicted probability band;
calculating a Wilson score interval for each predicted probability band based on the observed discharge rate and a number of historical patients within the predicted probability band, the Wilson score interval including a lower confidence bound and an upper confidence bound;
storing the Wilson score interval for each predicted probability band in a data structure accessible to the patient management system during inference; and
displaying the calibrated probabilities and the associated confidence intervals in the UI.

12. The method of claim 11, wherein displaying the calibrated probabilities and the associated confidence intervals in the UI comprises:
displaying a confidence interval display element including a point estimate representing an observed discharge rate for a predicted probability band corresponding to a selected probability, a lower confidence bound derived from the Wilson score interval for the predicted probability band, an upper confidence bound derived from the Wilson score interval for the predicted probability band, and a graphical confidence range indicator depicting a span between the lower confidence bound and the upper confidence bound with the point estimate indicated at a position within the span.

13. A method for a patient discharge date prediction system, comprising:
receiving, at the patient discharge date prediction system, secure feeds from one or more electronic health record databases of a hospital network;
receiving operational workflow data from one or more operational systems of a healthcare facility;
encoding the received patient data and the received operational workflow data into data vectors;
receiving a selected confidence level from a user;
predicting, for each patient of a first set of patients associated with the healthcare facility, a predicted discharge date and a discharge horizon probability distribution indicating a first probability that the patient will be discharged on a specified date, a second probability that the patient will be discharged the day after the specified date, and a third probability that the patient will be discharged two or more days from the specified date using a trained length of stay (LOS) prediction model based on the encoded data vectors, the trained LOS prediction model trained based on historical patient data;
predicting, for each patient of the first set of patients, a predicted discharge date window based on the trained LOS prediction model, the encoded data vectors, and the selected confidence level;
displaying a graphical user interface (GUI) that displays, for each patient of the first set of patients, the predicted discharge date, the predicted discharge date window of the patient, and the discharge horizon probability distribution; and
wherein training the LOS prediction model comprises: dividing the historical patient data into a plurality of groupings based on length of stay ranges; classifying each patient of the historical patient data into one of the plurality of groupings; generating data vectors for each patient, each data vector encoding patient data of the respective patient; training the LOS prediction model sequentially on each grouping using the data vectors of patients in that grouping and actual length of stay values; determining an accuracy rating for each grouping based on prediction performance of the LOS prediction model for that grouping; reordering the plurality of groupings based on the accuracy ratings; and retraining the LOS prediction model on the reordered groupings; and
wherein a width of each of the predicted discharge date window is at least partially based on the accuracy ratings.

14. The method of claim 13, further comprising:
predicting, based on the encoded data vectors and using the trained LOS prediction model, a time-of-day discharge probability distribution indicating a fourth probability that the patient will be discharged within a first time window on a discharge day, a fifth probability that the patient will be discharged within a second time window on the discharge day, and a sixth probability that the patient will be discharged within a third time window on the discharge day; and
displaying the predicted date for discharging the patient, the discharge horizon probability distribution, and the time-of-day discharge probability distribution in the GUI.

15. A patient discharge date prediction system, comprising:
one or more processors; and
memory storing instructions executable by the one or more processors to:
receive, at the patient discharge date prediction system, a secure feed from one or more electronic health record databases of a hospital network, the secure feed including patient data of a plurality of patients;
receive operational workflow data from one or more operational systems of the hospital;
output, for display on a display device, a graphical user interface (GUI) that includes a discharge date and a discharge horizon probability distribution of each patient of the plurality of patients predicted by a length of stay (LOS) prediction model based on an encoding of both of the patient data of the patient included in the secure feed and the operational workflow data, the LOS prediction model trained on de-identified historical patient data of the one or more electronic health record databases;
wherein the discharge horizon probability distribution indicates a first probability that the patient will be discharged on a current date, a second probability that the patient will be discharged the day after the current date, and a third probability that the patient will be discharged two or more days after the current date; and
wherein the LOS prediction model is a convolution neural network (CNN), and the LOS prediction model is trained by: dividing the de-identified historical patient data into a plurality of groupings based on length of stay ranges; classifying each patient of the de-identified historical patient data into one of the plurality of groupings; generating data vectors for each patient, each data vector encoding patient data of the respective patient; training the LOS prediction model sequentially on each grouping using the data vectors of patients in that grouping and actual length of stay values; determining an accuracy rating for each grouping based on prediction performance of the LOS prediction model for that grouping; reordering the plurality of groupings based on the accuracy ratings; and retraining the LOS prediction model on the reordered groupings.

16. The patient discharge date prediction system of claim 15, wherein further instructions are stored in the memory that when executed, cause the one or more processors to:
receive a selected confidence level from a user;
predict, for each patient of the plurality of patients, a discharge date window based on the trained LOS prediction model, the encoded data vectors, and the selected confidence level; and
display the discharge date window in the GUI;
wherein the predicted discharge date window is a window of time greater than a calendar day.

17. The patient discharge date prediction system of claim 15, wherein further instructions are stored in the memory that when executed, cause the one or more processors to:
apply Wilson score interval calibration to the discharge horizon probability distribution based on historical observed discharge rates of the hospital network to generate calibrated probabilities and associated confidence intervals for each probability of the discharge horizon probability distribution, wherein applying Wilson score interval calibration comprises:
binning historical patients into a plurality of predicted probability bands based on predicted probabilities generated by the LOS prediction model for the historical patients;
computing an observed discharge rate within each predicted probability band based on actual discharge outcomes of the historical patients within the predicted probability band;
calculating a Wilson score interval for each predicted probability band based on the observed discharge rate and a number of historical patients within the predicted probability band, the Wilson score interval including a lower confidence bound and an upper confidence bound; and
storing the Wilson score interval for each predicted probability band in a data structure accessible to the patient discharge date prediction system during inference; and
display the calibrated probabilities and the associated confidence intervals in the GUI.

18. The patient discharge date prediction system of claim 17, wherein the data structure is a lookup table, and wherein further instructions are stored in the memory that when executed, cause the one or more processors to periodically recalibrate the Wilson score intervals by:
accumulating additional historical discharge data subsequent to a previous calibration;
re-binning historical patients including patients associated with the additional historical discharge data into the plurality of predicted probability bands;
recomputing the observed discharge rate within each predicted probability band based on actual discharge outcomes of the historical patients including the patients associated with the additional historical discharge data;
recalculating the Wilson score interval for each predicted probability band based on the recomputed observed discharge rate and an updated number of historical patients within the predicted probability band; and
replacing previously stored Wilson score intervals in the lookup table with the recalculated Wilson score intervals.

19. The patient discharge date prediction system of claim 15, wherein further instructions are stored in the memory that when executed, cause the one or more processors to:
predict, based on the encoding of both of the patient data and the operational workflow data, using the LOS prediction model, a time-of-day discharge probability distribution indicating a fourth probability that the patient will be discharged within a first time window on a discharge day, a fifth probability that the patient will be discharged within an afternoon time window on the discharge day, and a sixth probability that the patient will be discharged within a third time window on the discharge day;
wherein the LOS prediction model generates the discharge date, the discharge horizon probability distribution, and the time-of-day discharge probability distribution simultaneously during a single forward pass of the encoding through the CNN; and
display the time-of-day discharge probability distribution in the GUI.

20. The patient discharge date prediction system of claim 19, wherein further instructions are stored in the memory that when executed, cause the one or more processors to:
in response to determining that the first probability of the discharge horizon probability distribution indicating that the patient will be discharged on the current date exceeds a predetermined threshold probability, automatically initiate one or more discharge preparation workflows for the patient, the one or more discharge preparation workflows including one or more of:
generating a notification to one or more care providers of the patient indicating one or more administrative tasks to be performed prior to discharging the patient;
arranging transportation for the patient;
initiating paperwork associated with discharging the patient; and
generating a message to coordinate staffing, pharmacy, or post-acute placement services based on the time-of-day discharge probability distribution; and
in response to determining that a probability of the time-of-day discharge probability distribution indicating that the patient will be discharged within the first time window exceeds a second predetermined threshold probability, automatically initiate time-specific discharge coordination workflows including one or more of:
scheduling patient transport within the first time window;
notifying bed management of anticipated bed availability within the first time window; and
coordinating pharmacy medication reconciliation for completion prior to the first time window.
